(19) United States
(12) Patent Application Publication
(10) Pub. No.: US 2013/0164270 A1
(43) Pub. Date: Jun. 27, 2013

(54) POLYNUCLEOTIDES AND CONSTRUCTS ENCODING SFLT1-14 AND METHOD FOR EFFICIENT PROPAGATION AND EXPRESSION THEREOF

(71) Applicants: Eli Keshet, Jerusalem (IL); Shay Sela, Haifa (IL)

(72) Inventors: Eli Keshet, Jerusalem (IL); Shay Sela, Haifa (IL)

(73) Assignee: Yissum Research Developement Company of the Hebrew University of Jerusalem Ltd., Jerusalem (IL)

(21) Appl. No.: 13/387,626

(22) PCT Filed: Jul. 29, 2010
(86) PCT No.: PCT/IL2010/000615

§ 371 Date: Aug. 17, 2012

Related U.S. Application Data
(60) Provisional application No. 61/229,395, filed on Jul. 29, 2009.

(51) Int. Cl. C07K 14/71

(52) U.S. Cl. CPC C07K 14/71
USPC 424/93.21; 536/23.5; 435/320.1; 435/252.33; 514/44 R; 435/252.31; 435/325; 435/348; 435/349; 435/366; 435/352; 435/363; 424/93.2

(57) ABSTRACT

The present invention relates to engineered polynucleotides and constructs comprising nucleic acid sequences encoding a specific splice-variant (sFLT1-14) of the VEGFR family Flt-1, methods for efficient propagation and expression thereof and compositions and uses thereof. More particularly, the invention relates to isolated polynucleotides comprising a nucleic acid sequence coding for sFlt1-14 or any fragment thereof comprising the serine-rich C-terminus region of said sFlt1-14, wherein at least one of the TCA serine coding codons in said serine-rich C-terminus region of sFlt1-14 as encoded by the nucleic acid sequence of SEQ ID NO. 1, is replaced by any one of TCT, TCC, TCG, AGT, AGC. The invention further provides compositions and method of treating VEGF-associated medical conditions using the polynucleotides of the invention.

### FIELD OF THE INVENTION

The present invention relates to polynucleotides and constructs comprising a nucleic acid sequence encoding tracts of identical successive amino acid residues and to methods for propagation and expression thereof. More particularly, the invention relates to engineered polynucleotides and constructs comprising nucleic acid sequences encoding a specific splice-variant (sFLT1-14) of the VEGFR family Flt-1, methods for efficient propagation and expression thereof and compositions and uses thereof.

### BACKGROUND OF THE INVENTION

All publications mentioned throughout this application are fully incorporated herein by reference, including all references cited therein.

VEGF is a sub-family of growth factors, specifically the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature). In vitro, VEGF has been shown to stimulate endothelial cell mitogenesis and cell migration. VEGF also enhances microvascular permeability and is sometimes referred to as vascular permeability factor. As an endothelial-specific mitogen, VEGF plays a key role in promoting both vasculogenesis and angiogenesis.

Angiogenesis is particularly important in the development of solid tumors. While there are more than 100 distinct types of cancer (and considerable heterogeneity within each tumor type), the mechanisms that fuel tumor growth and survival are relatively similar. Across most-if not all-malignancies, sustained angiogenesis is considered to be one of these central “hallmarks” of cancer.

A tumor needs an independent blood supply, which is acquired by the over-expression of growth factors that recruit new vasculature from existing blood vessels. The disruption of the delicate balance of pro- and anti-angiogenic factors, which is often referred to as the angiogenic switch, results in the creation and maintenance of a growing vascular network. While numerous pro-angiogenic factors have been characterized, for example angiopoietin-1 and basic fibroblast growth factor (bFGF), the VEGF ligand has been identified as the predominant regulator of tumor angiogenesis.

The VEGF ligand may affect tumor vasculature in three essential ways. Early in tumor development, VEGF may help new vasculature establish. Specifically, VEGF has been shown to stimulate tumor growth at both primary and metastatic sites through the recruitment of bone-marrow-derived progenitor cells that form the building blocks of a new vascular network. As this network develops, VEGF may continue to help new vasculature grow, providing the blood supply needed to drive further tumor growth and metastasis. Throughout tumor development, VEGF may also help existing vasculature survive, allowing tumors to sustain their metabolic requirements over their entire life cycle.

All members of the VEGF family stimulate cellular responses by binding to tyrosine kinase receptors (the VEGFRs) on the cell surface, causing them to dimerize and become activated through transphosphorylation. The VEGF receptors have an extracellular portion consisting of seven immunoglobulin-like domains, a single transmembrane spanning region and an intracellular portion containing a split tyrosine-kinase domain.

The activities of VEGF are mediated primarily by its interaction with two high-affinity receptor tyrosine kinases: fins-like tyrosine kinase-1 (FIt-I/VEGFR-1) and kinase-insert domain region (KDR/Flk-1NEGFR-2) both of which are expressed on vascular endothelial cell surfaces. FIt-I (VEGFR-1) may act as a dummy/decoy receptor, sequestering VEGF from VEGFR-2 binding, which appears to be particularly important during vasculogenesis in the embryo. Oncogene FLT belongs to the src gene family and is related to oncogene ROS (MIM 165020). Like other members of this family, it shows tyrosine protein kinase activity that is important for the control of cell proliferation and differentiation. The sequence structure of the FLT gene resembles that of the FMS gene (MIM 164770); hence, the name FLT was proposed as an acronym for FMS-like tyrosine kinase.

Alternative splicing of FIt-I results in the production of an endogenously secreted protein referred to as soluble Flt1 (sFlt1), which lacks the cytoplasmic and transmembrane domains but retains the ligand-binding domain. WO2008075363 that is a previous application of the present inventors, concerns a splice variant of the Flt1-1, being soluble and expressing a segment from Flt1's intron 14 (as well as exon 14), called sFlt1-14.

The inventors have previously demonstrated that the increased production of soluble VEGF receptors during pregnancy is entirely attributable to induced expression of placental sFlt1-14 starting by the end of the first trimester. Expression is dramatically elevated in the placenta of women with preeclampsia, specifically induced in abnormal clusters of degenerative syncytiotrophoblasts known as syncytial knots, where it may undergo further messenger RNA editing. sFlt1-14 is the predominant VEGF-inhibiting protein produced by the preeclamptic placenta, accumulates in the circulation, and hence is capable of neutralizing VEGF in distant organs affected in preeclampsia.

The inventors have tried to produce a plasmid coding for sFlt1-14 of WO2008075363 in bacteria and failed to produce any plasmid using the native sequence.

The inventors postulate that the cause of the failure was due to replication slippage in bacteria caused by the TCA tandem repeats coding for the polyserine tract at the C′ terminus of sFlt1-14.

The process of replication slippage involves the slipping of DNA polymerase III from the DNA template strand at a codon repeat region and the subsequent reattachment at a more distant site.

Misalignment of two DNA strands during replication can lead to DNA rearrangements such as deletions or duplications of varying lengths ranging from several nucleotides to entire genes. This process, also termed “copy-choice recombination”, has been suspected for a long time to occur both in prokaryotes and eukaryotes between repeated DNA sequences. The process is thought to encompass the following steps: (i) copying of the first duplication by the replication machinery, (ii) replication pausing and dissociation of the polymerase from the newly synthesized end, (iii) unpairing of the newly synthesized strand and its pairing with the second duplication, and (iv) resumption of the DNA synthesis. A heteroduplex is thus formed, containing one parental and one recombinant strand, which are separated by a second round of replication.

Replication slippage has been widely proposed as a probable mechanism of genome rearrangements, such as deletions between short duplications in bacteria, yeast, and mammalian mitochondria or deletions between long tandem repeats in Escherichia coli, as well as microsatellite instability.

The present invention discloses polynucleotides, constructs and methods for the efficient propagation and expression of nucleic acid sequences encoding polypeptides containing tracts of more than one successive identical amino acid residue, by the use of alternating codons encoding said successive identical amino acids. Thus, the polynucleotides, constructs and methods of the present invention facilitate the proper replication and propagation of sequences comprising consecutive identical codons, wherein said codons encode amino acids which may be encoded by at least two different codons, and, subsequently, the expression of said genes. Specifically, the invention permits the replication of constructs comprising the sFlt1-14 gene and the efficient expression of sFlt1-14.

It is therefore one object of the invention to provide an engineered polynucleotide encoding sFlt1-14 or any fragment thereof, wherein successive identical serine-encoding codons were altered to non-identical serine-encoding codons, allowing proper replication and expression of said polynucleotide.

Another object of the invention is to provide constructs comprising the engineered sFlt1-14 polynucleotides of the invention, which may be used for propagation and expression of said polynucleotides.

In yet another object the invention provides a method for the propagation of constructs encoding tracts of more than one successive identical amino acid encoded by identical nucleic acid codons, comprising the use of alternating codons encoding said successive identical amino acids.

These and other objects of the invention will become apparent as the description proceeds.

### SUMMARY OF THE INVENTION

According to a first aspect, the present invention provides an isolated polynucleotide comprising a nucleic acid sequence coding for sFlt1-14 or any fragment thereof comprising the serine-rich C-terminus region of said sFlt1-14. Within this isolated polynucleotide, at least one of the TCA serine coding codons in the serine-rich C-terminus region of sFlt1-14 as appear in the native sFlt1-14 nucleic acid sequence (SEQ ID NO. 1), is replaced by other serine coding codons, for example, any one of TCT, TCC, TCG, AGT, AGC.

In a second aspect, the present invention contemplates a nucleic acid construct comprising a nucleic acid sequence coding for sFlt1-14 or any fragment thereof comprising the serine-rich C-terminus region of sFlt1-14. At least one of the TCA serine coding codons in the serine-rich C-terminus region of sFlt1-14 as encoded by the nucleic acid sequence of SEQ ID NO. 1 is replaced by any one of TCT, TCC, TCG, AGT, AGC, and the construct optionally further comprises operably linked regulatory elements. The invention further provides an expression vector comprising the nucleic acid construct of the invention and host cell transformed or transfected with the expression vector of the invention.

According to a further aspect of the present invention, a pharmaceutical composition comprising the isolated polynucleotide according to the invention or any construct, expression vector or host cell comprising the same is provided. The composition further comprises a pharmaceutically acceptable carrier.

According to a further aspect, the invention discloses a method for the treatment of a VEGF-associated medical condition comprising the step of administering to a subject in need thereof a therapeutically effective amount of the isolated polynucleotide according to the invention, or any construct, expression vector, host cell or composition comprising the same.

In yet a further aspect, the invention contemplates a method for efficient propagation and expression of a nucleic acid sequence coding for sFlt1-14 or any fragment thereof comprising the serine-rich C-terminus region of sFlt1-14. The method comprises the step of providing a polynucleotide sequence encoding said sFlt1-14 or any construct or expression vector thereof, wherein at least one of the TCA serine coding codons in the serine-rich C-terminus region of sFlt1-14 as encoded by the nucleic acid sequence of SEQ ID NO. 1 is replaced by any one of TCT, TCC, TCG, AGT, and AGC.

These and other aspects of the invention will become apparent by the hand of the following figures.

### BRIEF DESCRIPTION OF THE FIGURES

FIG. 1A-1B. sFlt1-14 variant

The cDNA sequence encompassing the entire coding region of the natural sFlt1-14 (NM_{—}001160030.1, also denoted by SEQ ID NO.: 1) is shown in FIG. 1A-1B. The sequence and associated data presented are shown as provided by the NCBI Nucleotide database. The fragment encoded by intron 14 of sFlt1-14 is underlined.

FIG. 2. sFlt1-14 encoded protein

Amino acid sequence encompassing the entire coding region of the natural sFlt1-14 (NP_{—}001153502, also denoted by SEQ ID NO.: 2) is shown. The sequence and associated data presented are shown as provided by the NCBI Protein database. Residues encoded by exon 14 of sFlt1-14 are underlined, and those encoded by intron 14 of sFlt1-14 are emphasized in bold.

FIG. 3. sFlt1-14 engineered cDNA

The cDNA sequence (denoted by SEQ ID NO.: 3) encoding sFlt1-14 protein (NP_{—}001153502, also denoted by SEQ ID NO.: 2) is shown. The polyserine tract encoding region in intron 14 of sFlt1-14, where codons were engineered, is underlined.

FIG. 4. Expression of recombinant sFlt1 and sFlt1-14 proteins cDNAs encompassing the entire coding region of the natural sFlt1 (NP_{—}001153502.1) or the engineered sFlt1-14 of the invention (SEQ ID NOs.: 3) were sub-cloned into Bluescript expression vectors and transfected onto T7 polymerase-expressing HeLa cells. 24 hours later the cells were harvested. Proteins were detected by immunoblotting using the ab9540 antibody, directed against the extracellular domain of both isoforms, or by the CESS antibody targeting a specific C′ terminus fragment (SEQ ID NO. 21) of sFlt1-14 encoded by intron-14, which contains the poly-serine tract.

Abbreviations: Extra. Ab. (extracellular antibody; Ab9540); kD (kilo Dalton).

FIG. 5. Examples of fragments of the sFlt1-14 serine-rich C′-terminus region

The nucleotide and amino acid sequences of both the native and engineered sFlt1-14 are shown. (i), (ii), (iii) and (iv) mark the starting codon and amino acid for fragments of the native sFlt1-14 serine-rich C′-terminus region (amino acid sequences are denoted by SEQ ID NO. 7, 8, 9, 10). The native nucleic acid sequences in said fragments are denoted by SEQ ID NOs.: 11, 12, 13 and 14, respectively, and (v), (vi), (vii) and (viii) mark the starting codon and amino acid for fragments of the engineered sFlt1-14 serine-rich C′-terminus region (the nucleic acid sequences in said fragments are also denoted by SEQ ID NOs.: 15, 16, 17 and 18, respectively).

Abbreviations: Nat. sFlt1-14 (native sFlt1-14); Eng. sFlt1-14 (engineered sFlt1-14).

### DETAILED DESCRIPTION OF THE INVENTION

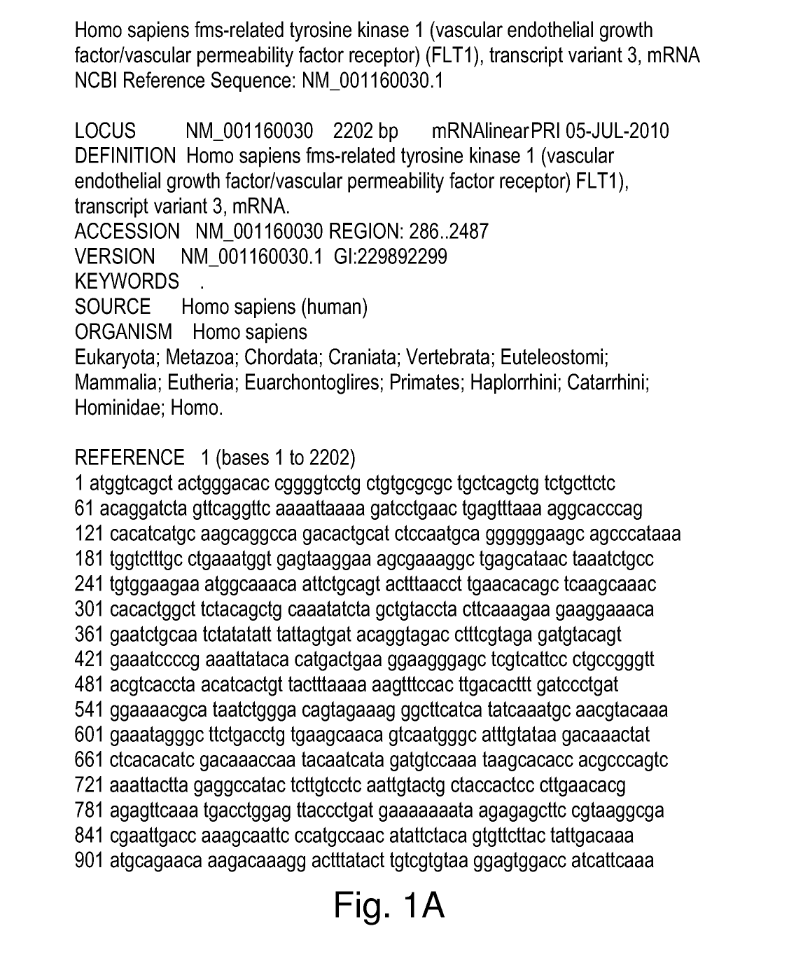

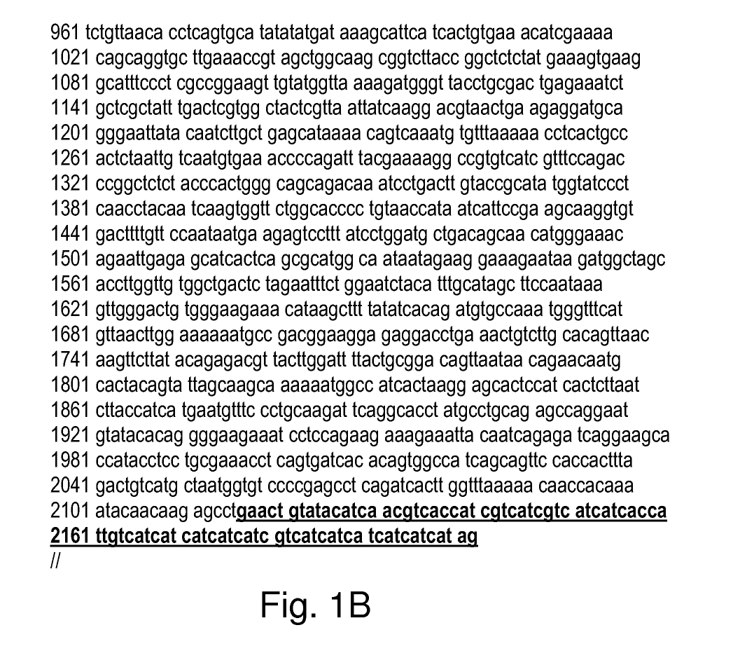

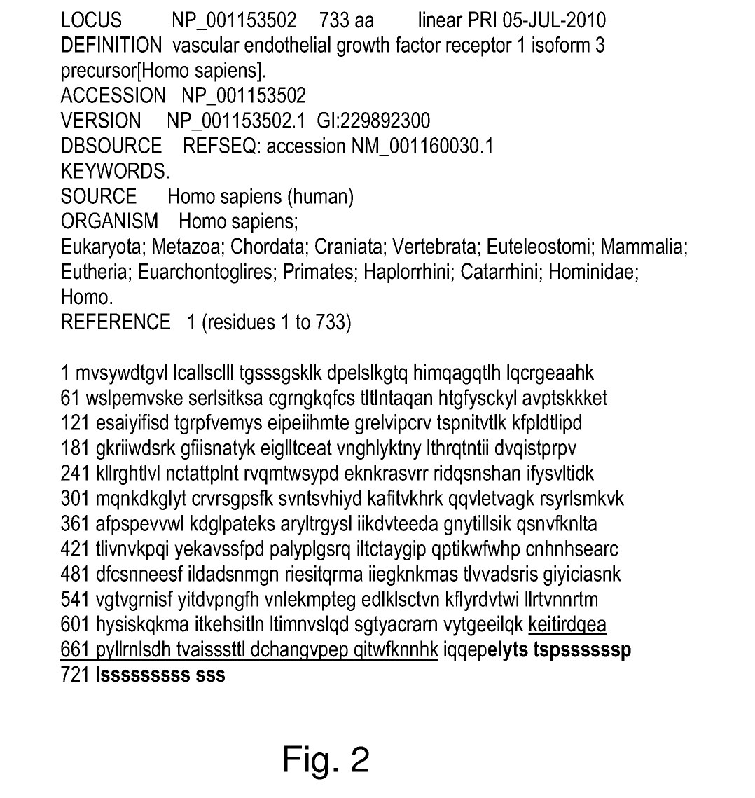

The present invention, in some embodiments thereof, relates to an isolated polynucleotide comprising a nucleic acid sequence coding for sFlt1-14. The polynucleotide of the invention comprises changes in nucleotide sequences encoding serine residues. The invention further provides nucleic acids constructs, vectors, host cells expressing said constructs or vectors, pharmaceutical compositions comprising the same and methods using the polynucleotide of the invention or any fragments thereof for the treatment of VEGF-associated medical conditions. The invention is also directed to a method of propagation and efficient expression of polynucleotides comprising repeat regions. In some embodiments, the repeat regions consist of at least two successive identical codons, the codons encode an amino acid which may be encoded by at least two different codons. The amino acids which may be encoded by at least two different codons are serine, arginine, leucine, valine, praline, alanine, threonine, glycine, isoleucine, phenylalanine, tyrosine, histidine, glutamine, asparagine, lysine, aspartic acid, glutamic acid and cysteine. More specifically, the present invention relates to a method of propagation of polynucleotides comprising repeat regions, as described above, encoding polypeptides which are useful for the diagnosis and treatment of VEGF-associated medical conditions and expression thereof. In particular, the present invention is directed to a method of propagation of polynucleotides encoding Flt-1 splice variant, sFlt1-14 (denoted by SEQ ID NO.:1 and illustrated by FIG. 1A-1B). Examples of genomic Flt1 are depicted in GeneBank Accession No. NC_{—}000013.9 region: complement (27773790 to 27967232) GI:51511729 for human genomic Flt1 and GeneBank Accession No. NC_{—}006480.2 region: complement (27975879 to 28168596) GI: 114795054 for chimpanzee genomic Flt1.

The phrase “splice variant”, as used herein, refers to alternative forms of RNA transcribed from a VEGF receptor gene. Splice variation arises naturally through use of alternative splicing sites within a transcribed RNA molecule, or less commonly between separately transcribed RNA molecules, and may result in several different mRNAs transcribed from the same gene. Splice variants may encode polypeptides having altered amino acid sequence due to intron inclusion, exon exclusion or a combination of both. The term splice variant is also used herein to denote a polypeptide encoded by a splice variant of an mRNA transcribed from a gene.

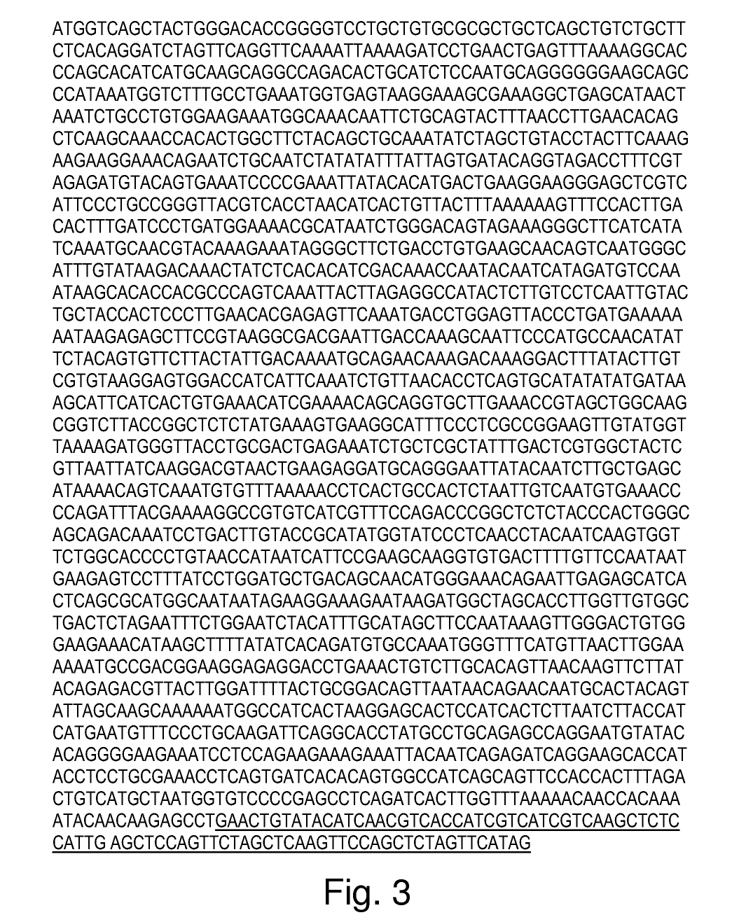

The splice variant sFlt1-14 (denoted by the nucleic acid sequence of SEQ ID NO.:1, encoding the polypeptide of SEQ ID NO. 2, that is the sFlt1-14 polypeptide) comprises a C′-terminal polyserine tract encoded by consecutive identical serine codons. As illustrated in Example 1, the repetitive codon sequence interfered with proper replication of the construct comprising it, probably as a result of DNA polymerase slippage. The present invention is based on the realization that since serine is coded by six different codons, they can be used alternatively in order to avoid slippage. Thus, the invention provides an engineered sFlt1-14 polynucleotide (particular example shown by FIG. 3), wherein successive identical serine codons were exchanged with alternative serine codons, thus preventing said slippage and facilitating proper replication of construct.

The term “engineered”, “engineered polynucleotide” or “engineered sequence”, as referred to herein, relates to a polynucleotide sequence that has been modified using molecular methods known in the art. Such modifications comprise deletions of single or multiple nucleotides, C′- or N′-terminal truncation, single or multiple nucleotide replacements, fusion of any of the above to other single-, oligo- or polynucleotides, or any combination of the aforementioned. Although the term “engineered” may also generally be applied to peptide or polypeptide sequences that are modified by the above modifications of the peptide- or polypeptide-encoding polynucleotide sequence, in the context of the present application the various polynucleotide sequence modifications described preserve the native peptide or polypeptide sequence, and accordingly the term “engineered” relates to the polynucleotide sequence, rather than the peptide or polypeptide sequence.

The term “DNA polymerase slippage”, “polymerase slippage” or “slippage” as used herein relates to a process known as replication slippage or copy-choice recombination. The slippage occurs between repeated DNA sequences in both prokaryotes and eukaryotes. Slippage involves DNA polymerase pausing, which must take place within the direct repeat, and that the pausing polymerase dissociates from the DNA. Upon polymerase dissociation, only the terminal portion of the newly synthesized strand separates from the template and anneals to another direct repeat. Resumption of DNA replication then completes the slippage process. The present invention thus demonstrates a novel strategy for eliminating propagation and expression deficiencies of sequences containing repeating residues caused by such slippage. By providing an engineered polynucleotide with reduced repetitive codons, specifically, devoid of repetitive codons, the invention eliminates slippage and thereby enables efficient replication of constructs containing such polynucleotides.

Thus, according to a first aspect, the present invention provides an isolated polynucleotide comprising a nucleic acid sequence coding for sFlt1-14 or any fragment thereof comprising the serine-rich C-terminus region of said sFlt1-14. Within this isolated polynucleotide, at least one of the TCA serine coding codons in the serine-rich C-terminus region of sFlt1-14 as appear in the native sFlt1-14 nucleic acid sequence (SEQ ID NO. 1), is replaced by other serine coding codons, for example, any one of TCT, TCC, TCG, AGT, AGC.

The term “serine-rich C-terminus”, refers to the C-terminal part of the sFlt1-14 splice variant, specifically, to the terminal part encoded by intron-14, or any fragment thereof. This terminal region (SEQ ID NO. 4) comprises 20 serine residues out of a total of 28 amino acid residues (about 70%). The invention therefore relates to any fragment or part of this C-terminal region of sFlt1-14 molecule that contains about 10% to 100% serine residues, specifically, 20% to 90%, 30% to 80%, 40% to 70%, or 50% to 60% serine residues. According to more particular embodiments, a fragment of the “serine-rich C-terminal region of the sFlt1-14 splice variant” according to the invention may contain about 10%, 15%, 20%, 25%, 30%, 35%, 40%, 45%, 50%, 55%, 60%, 65%, 70%, 75%, 80%, 85%, 90%, 95% or 100% serine residues.

As used herein, the term “fragments” refers to parts of the sFlt1-14 protein comprising at least 6, preferably at least 7, preferably at least 8, preferably at least 9, preferably at least 10, preferably at least 11, preferably at least 12, preferably at least 13, preferably at least 14, preferably at least 15, preferably at least 16, preferably at least 17, preferably at least 18, preferably at least 19, preferably at least 20, preferably at least 21, preferably at least 22, preferably at least 23, preferably at least 24, preferably at least 25, preferably at least 26, preferably at least 27, or most preferably 28 continuous amino acids encoded by intron 14 as denoted by SEQ ID NO. 4. Preferably, the fragment includes the region that contains several serine residues present in tandem. Non limiting examples of such fragments may include any one of SEQ ID NO. 7 to 10 and 24, 26, 28, 30, 32, 34, 36, 38, 40 and 42.

It should be further appreciated that as used herein in the specification and in the claims section below, the term “C′-terminus region” refers to a continuous or discontinuous sequences involving amino acids derived from any location or locations, either continuous or dispersed, along the 100 C′-terminal amino acids, preferably, the 28 C′-terminal amino acids of sFlt1-14, as appropriate. Continuous or discontinuous sequence typically includes 3-8 continuous or discontinuous amino acids. The term “amino acid” or “amino acids” is understood to include the 20 naturally occurring amino acids; those amino acids often modified post-translationally in vivo, including, for example, hydroxyproline, phosphoserine, and phosphothreonine; and other less common amino acids, including but not limited to 2-aminoadipic acid, hydroxylysine, isodesmosine, nor-valine, nor-leucine, and ornithine. Furthermore, the term “amino acid” includes both D- and L-amino acids.

It should be understood that the described serine-rich region may be any C′-terminal fragment of sFlt1-14, the amino acid sequence of sFlt1-14 also denoted as SEQ ID NO.: 2, wherein the fragment comprises sFlt1-14 amino acid residues in positions 706 to 733 (SEQ ID NO. 4) according to SEQ ID NO.: 2. In one specific embodiment, such fragment may comprise the C′ terminal region that encoded by intron-14, specifically, such fragment includes the amino acid sequence from positions 707 to 733. It should be noted that shorter fragments are also encompassed by the invention, e.g., positions 708 to 733, positions 709 to 733, positions 710 to 733, positions 711 to 733, positions 712 to 733, positions 713 to 733, positions 714 to 733, positions 715 to 733, positions 716 to 733, positions 717 to 733, positions 718 to 733, positions 719 to 733, positions 720 to 733, positions 721 to 733, positions 722 to 733, positions 723 to 733, positions 724 to 733, positions 725 to 733, positions 726 to 733 or positions 727 to 733.

Thus, the polynucleotide of the invention comprises a serine-rich C-terminus region of sFlt1-14 wherein at least one TCA codon replaced by any one of TCT, TCC, TCG, AGT, AGC. The at least one TCA codon may be selected from the TCA codons encoding the serine residues in positions 710, 712, 715, 717-719, 722-726 or 728-733, said amino acid positions are numbered according to their positions in SEQ ID NO.:2. It should be further noted that the invention further encompasses polynucleotide molecule, where other serine codons, for example the TCG codon, that appear in the original native sFlt1-14 sequence (SEQ ID NO. 1), are replaced by any one of TCT, TCC, TCA, AGT or AGC. In any case, the peptide sequence encoded by the nucleotide will not be changed by these replacements.

As indicated above, the invention provides a polynucleotide comprising nucleic acid sequence. The term “nucleic acid” or “polynucleotides” as used herein refer to a macromolecule composed of chains of monomeric nucleotides. The most common nucleic acids are deoxyribonucleic acid (DNA) and ribonucleic acid (RNA). Each nucleotide consists of three components: a nitrogenous heterocyclic base, which is either a purine or a pyrimidine; a pentose sugar; and a phosphate group. Nucleic acid types differ in the structure of the sugar in their nucleotides—DNA contains 2-deoxyribose while RNA contains ribose, the only difference between the two being the presence of a hydroxyl group. Also, the nitrogenous bases found in the two nucleic acid types are different: adenine (A), cytosine (C), and guanine (G) are found in both RNA and DNA, while thymine (T) only occurs in DNA and uracil (U) only occurs in RNA. Other rare nucleic acid bases can occur, for example inosine in strands of mature transfer RNA. Nucleic acids are usually either single-stranded or double-stranded, though structures with three or more strands can form. A double-stranded nucleic acid consists of two single-stranded nucleic acids held together by hydrogen bonds, such as in the DNA double helix. In contrast, RNA is usually single-stranded, but any given strand may fold back upon itself to form secondary structure as in tRNA and rRNA.

The polynucleotide of the invention comprises changes in the serine coding codons. The term “codon” as referred to herein relates to a sequence of three adjacent nucleotides, which encode for a specific amino acid during protein synthesis, or translation, with exception of three codons, called “stop codons,” which signal protein synthesis to terminate. The genome of an organism is inscribed in DNA, or in the case of some viruses, RNA. The portion of the genome that codes for a protein or an RNA is referred to as a gene. Those genes that code for proteins are composed of tri-nucleotide units called codons, each coding for a single amino acid.

Each protein-coding gene is transcribed into a template molecule of the related polymer RNA, known as messenger RNA or mRNA. This, in turn, is translated on the ribosome into an amino acid chain or polypeptide. The process of translation requires transfer RNAs specific for individual amino acids with the amino acids covalently attached to them, guanosine triphosphate as an energy source, and a number of translation factors. tRNAs have anticodons complementary to the codons in mRNA and can be “charged” covalently with amino acids at their 3′ terminal CCA ends. Individual tRNAs are charged with specific amino acids by enzymes known as aminoacyl tRNA synthetases, which have high specificity for both their cognate amino acids and tRNAs. The high specificity of these enzymes is a major reason why the fidelity of protein translation is maintained.

There are 4³=64 different codon combinations possible with a triplet codon of three nucleotides; all 64 codons are assigned for either amino acids or stop signals during translation. The standard genetic code shown in Table 1 shows what amino acid each of the 64 codons specifies.

As indicated above, in certain embodiments and aspects, the invention provides isolated and purified polynucleotides. As used herein, “isolated” or “substantially purified”, in the context of a nucleic acid molecule encoding a polypeptide, such as the sFlt1-14 protein, as exemplified by the invention, means the nucleic acid sequence has been removed from its natural milieu or has been altered from its natural state. As such “isolated” does not necessarily reflect the extent to which the nucleic acid molecule has been purified. However, it will be understood that a nucleic acid molecule that has been purified to some degree is “isolated”. If the nucleic acid molecule does not exist in a natural milieu, i.e. it does not exist in nature, the molecule is “isolated” regardless of where it is present. By way of example, a polynucleotide that does not naturally exist in humans is “isolated” even when it is present in humans.

Furthermore, the term “isolated” or “substantially purified”, when applied to a nucleic acid or protein, denotes that the nucleic acid or protein is essentially free of other cellular components with which it is associated in the natural state. It is preferably in a homogeneous state, although it can be in either a dry or aqueous solution. Purity and homogeneity are typically determined using analytical chemistry techniques such as polyacrylamide gel electrophoresis or high performance liquid chromatography. A polynucleotide which is the predominant species present in a preparation is substantially purified.

As indicated herein, the C-terminal region of sFlt1-14 is preferably the part encoded by intron-14. The term “intron” refers to a DNA sequence present in a gene which is not usually translated into protein and is generally found between exons. These sections are transcribed to precursor mRNA (pre-mRNA) and some other RNAs (such as long noncoding RNAs), and subsequently removed by splicing during the processing to mature RNA. However, in the specific case of sFlt1-14, the sequence that was formerly regarded as intron 14 (SEQ ID NO.: 5) was found to be, in fact, expressed as part of the sFlt1-14 polypeptide, when the specific splicing events which create sFlt1-14 occur. The term “exon”, as referred to herein, relates to a nucleic acid sequence that is represented in the mature form of an RNA molecule after either portions of a precursor RNA (introns) have been removed by cis-splicing. The mature RNA molecule can be a messenger RNA or a functional form of a non-coding RNA such as rRNA or tRNA. Depending on the context, exon can refer to the sequence in the DNA or its RNA transcript. In the context of the present application, the exon is transcribed to mature mRNA. “cis-splicing”, as referred to herein, relates to a modification of RNA after transcription, in which introns are removed and exons are joined. This is needed for the typical eukaryotic messenger RNA before it can be used to produce a correct protein through translation. For many eukaryotic introns, splicing is done in a series of reactions which are catalyzed by the spliceosome, a complex of small nuclear ribonucleoproteins (snRNPs), but there are also self-splicing introns.

In order to allow appropriate replication and efficient expression of the polynucleotide of the invention, preferably, by avoiding slippage, it is desired that at least part of the serine coding codons will be either followed or preceded or both followed and preceded by non-identical serine coding codons.

For efficient propagation and expression of the sFlt1-14 molecule, the nucleic acid sequence encoding said protein is engineered by replacing at least part of the identical serine coding codons originally comprise within the native nucleic acid sequence.

In specific embodiments, at least thirteen of the serine coding codons comprised within the serine-rich C-terminus encoding sequence of the polynucleotide of the invention are at least one of followed, preceded or both by a non-identical codon while coding for the same amino acid sequence as encoded by SEQ ID NO 1. The term “non-identical codon” as used herein refers to a codon coding any amino acid residue or any non-identical serine coding codon.

It will be appreciated that according to some embodiments, at least thirteen of the serine coding codons comprised within the maximal serine-rich C-terminus encoding sequence denoted by the original nucleic acid sequence encoding sFlt1-14 (SEQ ID NO. 1, or in the “intron-14” region thereof as denoted by SEQ ID NO.: 5) are either followed, preceded or both, followed and preceded, by a non-identical codon. It is noteworthy that the “non-identical codon” may be any codon, and not necessarily a serine codon. More specifically, “non-identical codon” includes a non-identical serine codon, a stop codon, or a codon encoding another amino acid residue that appears in the original native sFlt1-14 nucleic acid sequence as denoted by SEQ ID NO. 1 (and also in the “intron-14” fragment thereof as denoted by SEQ ID NO. 5), encoding the amino acid sequence of sFlt1-14 (SEQ ID NO. 2). This is the case, for example, for codons encoding serine residues in positions 710, 712, 714, 719, 722 and 733, wherein said amino acid (positions are numbered according to their positions in SEQ ID NO.:2), are either followed, preceded or both by a non-serine coding codon (i.e., codons coding for different amino acid residues appearing in the original sequence or a stop codon). More specifically, for example, the serine coding codon in the native sFlt1-14 nucleic acid sequence as denoted by SEQ ID NO. 1, encoding serine in position 710 is preceded by a Threonine coding codon (position 709). This serine coding codon is also followed by a Threonine coding codon (for Threonine in position 711). The native serine coding codon of serine 719, is preceded by an identical serine coding codon (TCA, encoding serine 718) but is followed by a proline coding codon (proline in position 720). As indicated above, “non-identical codon” as used herein also encompasses stop codons, for example the stop codon following the serine residue in position 733.

Still further, the native polynucleotide sequence encoding sFlt1-14 nucleic acid sequence as denoted by SEQ ID NO. 1, also includes serine coding codons that are followed or preceded or both, by a different serine coding codons, for example, the serine coding codons of serine residues in positions 715, 716, 717, 726, 727 and 728. For example, the codon encoding serine residue in position 716 in the native polypeptide is TCG. This codon is followed by a non-identical serine coding codon (TCA, encoding serine in position 717) and is also preceded by a non-identical serine coding codon (TCA, encoding serine in position 715). The native polynucleotide sequence of SEQ ID NO. 1, therefore includes 12 serine codons that are either followed or preceded or followed and preceded by a non-identical codon that are serine residues in positions 710, 712, 714, 715, 716, 717, 719, 722, 726, 727, 728 and 733.

In practice, such embodiments dictate a minimal change of at least one serine coding codon in comparison with the codons presented in sFlt1-14 nucleic acid sequence denoted by SEQ ID NO.:1, in any one of the following serine coding codons of positions 718, 723, 725, 729, 730, 731 and 732, thus creating a sequence where at least 13 serine codon are either preceded or followed by a non-identical codon.

According to another specific embodiment, the invention provides an isolated polynucleotide having no more than 5 identical serine coding codons in tandem. Preferably, the different serine coding codons are distributed so that no more than 5, preferably no more than 4, more preferably no more than 3, most preferably no more than 2 identical serine coding codons are present in tandem (either followed, preceded or both). The term “in tandem”, as used herein, refers to an arrangement of two or more objects placed one next to the other, specifically, either nucleotides, nucleotide triplets constituting codons, or amino acids, in consecutive positions within a sequence. For example, the present invention is directed to a serine-rich C′-terminal region of sFlt1-14, where some of the aforesaid serine residues are arranged in tandem, that is, several serine residues are positioned consecutively in sequence. It will be appreciated that a series of identical amino acids arranged in tandem may be encoded by different codons and each codon may be composed of different nucleotides, thus “in tandem” should be understood as relating to either nucleotides, nucleotide triplets constituting codons, or amino acids, as appropriate, but not necessarily to all combinations thereof, for a given sequence.

According to certain embodiments, at least 1, at least 2, at least 3, at least 4, at least 5, at least 6, at least 7, at least 8, at least 9, at least 10, at least 11, at least 12, at least 13, at least 14, at least 15, at least 16 or at least 17 of the TCA serine coding codons in said serine-rich C-terminus region of sFlt1-14 as encoded by the native nucleic acid sequence as denoted by SEQ ID NO. 1, are replaced by any other (non-TCA) serine coding-codon, for example, any one of TCT, TCC, TCG, AGT, AGC.

In yet another specific embodiment, the invention provides an isolated polynucleotide having at least three of the TCA serine coding codons in said serine-rich C-terminus region of sFlt1-14 as encoded by the nucleic acid sequence of SEQ ID NO. 1, replaced by any one of TCT, TCC, TCG, AGT, AGC.

It should be further noted that the invention further encompasses polynucleotide molecule, where other serine codons, for example the TCG codon, that appear in the original native sFlt1-14 sequence (SEQ ID NO. 1), are replaced by any one of TCT, TCC, TCA, AGT or AGC.

In other embodiments, most of, and specifically, all, the serine coding codons comprised within the serine-rich C-terminus encoding sequence of the polynucleotide of the invention are either followed, preceded or both by a non-identical codon.

More particularly, each of the serine coding codons comprised within the original native sFlt1-14 nucleic acid sequence as denoted by SEQ ID NO. 1 (and also in the “intron-14” fragment thereof as denoted by SEQ ID NO. 5) are either followed, preceded or both by a non-identical codon, and the same principal applies to any serine-rich C′-terminus sFlt1-14 fragment. Similarly to other embodiments, the “non-identical codon” may be any codon, and not necessarily a serine codon. Such embodiments describe a polynucleotide sequence according to the invention, wherein there is a minimal change of at least four codons in comparison with the codons presented in the serine-rich sFlt1-14 C′-terminal fragment sequence denoted by SEQ ID NO.: 5, including codons in amino acid positions 718, 724, and a combination selected from 729 and 732, 730 and 732, and 730 and 733, said amino acid positions are numbered according to their positions in SEQ ID NO.:2, thus creating a sequence where all serine codon are either preceded or followed by a non-identical codon, while still coding for the amino acid sequence of SEQ ID No 2.

In yet another particular embodiments, at least one codon of each at least two successive serine coding codons comprised within the serine-rich C-terminus encoding sequence of the polynucleotide of the invention are either followed, preceded or both by a non-identical serine codon selected from the group consisting of TCA, TCT, TCC, TCG, AGT, AGC.

More specifically, in certain embodiments, at least one codon comprised in each pair of successive serine codons comprised within the serine-rich C′-terminus of the original native sFlt1-14 nucleic acid sequence as denoted by SEQ ID NO. 1 (and also in the “intron-14” fragment thereof as denoted by SEQ ID NO. 5) is either followed, preceded or both by a non-identical serine codon selected from the group consisting of TCA, TCT, TCC, TCG, AGT, AGC. According to these embodiments, a minimum of three codons must be replaced in the full serine-rich C′-terminus sFlt1-14 fragment denoted as SEQ ID NO.:5. These changes will not change the peptide encoded by the nucleotide sequence. The minimal changes include a change in the codons encoding serine residues in positions 731, one codon selected from positions 723 and 724, and one codon selected from positions 717-719, said amino acid positions are numbered according to their positions in SEQ ID NO.:2. It should be appreciated that since codon 731 must be replaced according to these embodiments, and seeing that codon 731 is TCA, these embodiments fall within the scope of the invention.

In more specific embodiments, each codon of each at least two successive serine coding codons comprised within the serine-rich C-terminus encoding sequence of the polynucleotide of the invention are either followed, preceded or both by a non-identical serine codon selected from the group consisting of TCA, TCT, TCC, TCG, AGT, AGC. More particularly, each codon comprised in each pair of successive serine codons comprised within the serine-rich C′-terminus of the original native sFlt1-14 nucleic acid sequence as denoted by SEQ ID NO. 1 (and also in the “intron-14” fragment thereof as denoted by SEQ ID NO. 5) is either followed, preceded or both by a non-identical serine codon selected from the group consisting of TCA, TCT, TCC, TCG, AGT, AGC. According to these specific embodiments, a minimum of six codons must be replaced in the full serine-rich C′-terminus sFlt1-14 fragment denoted as SEQ ID NO.:5. It should be indicated that these changes will not change the peptide encoded by the nucleotide sequence. A non limiting example for a polynucleotide sequence comprising said minimal changes is a polynucleotide having a change in the codons encoding serine residues in positions 718, 723, 725, 729, 731 and 733, said amino acid positions are numbered according to their positions in SEQ ID NO.:2. It should be appreciated that in this particular embodiment all the indicated serine residues are encoded by TCA, these embodiments fall within the scope of the invention.

It should be noted that serine is unique among the amino acids in that it has six codons, from 2 distinct groups: the TCN group (TCA, TCC, TCG, and TCT) and the AGY group (AGC and AGT). Transitions between a TCN codon and an AGY codon require a no synonymous intermediate, that is, a TCN-group codon cannot be transformed into an AGY-group codon via a single nucleotide exchange, but rather at least two nucleotides must be exchanged simultaneously to complete the transformation.

Thus, according to one particular embodiment, the polynucleotide of the invention may alternately comprise serine coding codons of the TCN group, more specifically, any one TCA, TCC, TCG, and TCT located next (either followed, preceded or both) to a serine coding codons of the AGY group, specifically, any one of AGC and AGT.

In yet more specific embodiments, each codon of at least two successive serine coding codons selected from the group consisting of TCA, TCT, TCC and TCG comprised within the serine-rich C-terminus encoding sequence of the polynucleotide of the invention are either followed, preceded or both by a non-identical serine codon selected from the group consisting of AGT and AGC.

In a preferred embodiment, the polynucleotide of the invention comprises a nucleic acid sequence at least 31% homologous to the serine-rich C-terminus encoding region of SEQ ID NO. 3.

According to one embodiment, the polynucleotide sequence of the invention demonstrates at least 70%, more preferably at least 72%, more preferably at least 74%, more preferably at least 76%, more preferably at least 78%, more preferably at least 80%, more preferably at least 82%, more preferably at least 84%, more preferably at least 86%, more preferably at least 88%, more preferably at least 90%, more preferably at least 92%, more preferably at least 94%, most preferably at least 96% homology to the complete engineered sFlt1-14 sequence as denoted by SEQ ID NO. 3, since intron 14 sequence constitutes only 4% of the complete sFlt1-14 coding sequence. The homology relates to the degree of identity between any full length homolog and the claimed engineered sequence, also denoted as SEQ ID NO. 3.

According to certain embodiments, the serine-rich C-terminus encoding region of any homolog must share at least 20 to 99% homology to the serine-rich C-terminus encoding region of the engineered polynucleotide of the invention, of SEQ ID NO. 3. More specifically, the serine-rich C-terminus encoding region of any homolog must share at least 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 96, 97, 98, 99% and more, homology to the serine-rich C-terminus encoding region of the engineered polynucleotide of the invention, of SEQ ID NO. 3. According to one particular embodiment, the serine-rich C-terminus encoding region of any homolog must share at least 31% homology to the serine-rich C-terminus encoding region of the engineered polynucleotide of the invention, of SEQ ID NO. 3.

Reference to “sequence identity” in relation to other sequences, be them amino acid or nucleotide sequences, is equivalent to amino acid or nucleic acid “homology”.

As used herein, amino acid or nucleic acid “identity” is equivalent to amino acid or nucleic acid “homology”. The percent identity between the two sequences is a function of the number of identical positions shared by the sequences, taking into account the number of gaps, and the length of each gap, which need to be introduced for optimal alignment of the two sequences.

The terms “identical”, “substantial identity”, “substantial homology” or percent “identity”, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region or over the entire molecule, as determined using BlastN software of the National Center of Biotechnology Information (NCBI) using default parameters.

“Homology” with respect to a specific polynucleotide sequence, for example, of SEQ ID NO. 3, and its functional derivatives is defined herein as the percentage of nucleotides in the candidate sequence that are identical or similar with nucleotides of a corresponding native polynucleotide, after aligning the sequences and introducing gaps, if necessary, to achieve the maximum percent homology, and not considering any conservative substitutions as part of the sequence identity. Neither N-nor C-terminal extensions nor insertions or deletions shall be construed as reducing identity or homology. Methods and computer programs for the alignment are well known. It should be appreciated that by the terms “insertions” or “deletions”, as used herein it is meant any addition or deletion, respectively, of nucleotides to the polynucleotide of the invention, of between 1 to 50 nucleotides, between 20 to 1 nucleotides and specifically, between 1 to 10 nucleotides. More particularly, insertions or deletions may be of any one of 1, 2, 3, 4, 5, 6, 7, 8, 9, or 10 nucleotides. It should be recognized that insertions or deletions may be additions or reduction of nucleotides from the 5′-, the 3′-end of the molecule or within the molecule and any combinations thereof.

It should be appreciated that in certain embodiments, any homolog of the polynucleotide of SEQ ID NO.3 is encompassed by the invention, particularly, homolog exhibiting between about 70 to 96% homology or more, specifically, 96% homology or more, provided that such polynucleotide in not a polynucleotide comprising, having or consisting of the nucleic acid sequence as denoted by SEQ ID NO. 1.

A specific example of the polynucleotide sequence of the invention (coding for the full sFlt1-14 of SEQ ID NO. 2) is provided in FIG. 3, where the engineered intron 14 is underlined (also denoted as SEQ ID NO. 3 and Example 1).

Thus, in a particular embodiment, the isolated polynucleotide of the invention comprises the nucleic acid sequence as denoted by SEQ ID NO. 3, or any fragment thereof encoding the serine-rich C-terminus region of sFlt1-14. Non-limiting examples for polynucleotides encoding fragments of the serine-rich C-terminus region of sFlt1-14 are denoted by any one of SEQ ID NO. 15 to 18 and SEQ ID NO. 23, 25, 27, 29, 31, 33, 35, 37, 39 and 41.

It should not be overlooked that the principle of the invention which facilitates replication and expression of identical amino acid tracts may be readily applied to other amino acids encoded by at least two different codons, such as arginine (encoded by CGT, CGC, CGA, CGG, AGA and AGG), leucine (encoded by TTA, TTG, CTT, CTC, CTA and CTG), valine (encoded by GTT, GTC, GTA and GTG), proline (encoded by CCT, CCC, CCA and CCG), alanine (encoded by GCT, GCC, GCA and GCG), threonine (encoded by ACT, ACC, ACA and ACG), glycine (encoded by GGT, GGC, GGA and GGG), isoleucine (encoded by ATT, ATC and ATA), phenylalanine (encoded by TTT and TTC), tyrosine (encoded by TAT and TAC), histidine (encoded by CAT and CAC), glutamine (encoded by CAA and CAG), asparagine (encoded by AAT and AAC), lysine (encoded by AAA and AAG), aspartic acid (encoded by GAT and GAC), glutamic acid (encoded by GAA and GAG) and cysteine (encoded by TGT and TGC) as well as serine (encoded by TCT, TCC, TCA, TCG, AGT and AGC). The codons encoding each amino acid are also presented in Table 1 below:

TABLE 1
The genetic code -nucleic acid triplet codes for amino acids

| Codon | AA | Codon | AA | Codon | AA | Codon | AA |
|---|---|---|---|---|---|---|---|
| TTT | Phe | TCT | Ser | TAT | Tyr | TGT | Cys |
| TTC | Phe | TCC | Ser | TAC | Tyr | TGC | Cys |
| TTA | Leu | TCA | Ser | TAA | Stop | TGA | Stop |
| TTG | Leu | TCG | Ser | TAG | Stop | TGG | Trp |
| CTT | Leu | CCT | Pro | CAT | His | CGT | Arg |
| CTC | Leu | CCC | Pro | CAC | His | CGC | Arg |
| CTA | Leu | CCA | Pro | CAA | Gln | CGA | Arg |
| CTG | Leu | CCG | Pro | CAG | Gln | CGG | Arg |
| ATT | Ile | ACT | Thr | AAT | Asn | AGT | Ser |
| ATC | Ile | ACC | Thr | AAC | Asn | AGC | Ser |
| ATA | Ile | ACA | Thr | AAA | Lys | AGA | Arg |
| ATG | Met | ACG | Thr | AAG | Lys | AGG | Arg |
| GTT | Val | GCT | Ala | GAT | Asp | GGT | Gly |
| GTC | Val | GCC | Ala | GAC | Asp | GGC | Gly |
| GTA | Val | GCA | Ala | GAA | Glu | GGA | Gly |
| GTG | Val | GCG | Ala | GAG | Glu | GGG | Gly |

It should be noted that the different nucleotides A, T, C and G indicated in each codon are the four nucleotides adenine, thymine, cytosine and guanine, respectively. AA as indicated in the Table represents amino acids. It should be further noted that the different amino acid residues are also indicated herein using the three letter code, for example, Phe (phenylalanine), Leu (leucine), Ile (isoleucine), Met (methionine), Ser (serine), Pro (proline), Thr (threonine), Ala (alanine), Tyr (tyrosine), His (histidine), Gln (glutamine), Asn (asparagine), Lys (lysine), Asp (aspartic acid), Glu (glutamic acid), Cys (cysteine), Trp (tryptophan), Arg (arginine), Gly (glycine). In specific embodiments, the polynucleotide sequence of the invention encodes the sFlt1-14, having a polyserine tract. The term “polyserine tract” as used herein refers to a peptide or polypeptide comprising at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, at least fifty-five, at least sixty, at least sixty-five, at least seventy, at least seventy-five, at least eighty, at least eighty-five, at least ninety, at least ninety-five, or at least one-hundred consecutive serine residues, preferably at least twelve consecutive serine residues, wherein said identical residues are encoded by identical codons.

More generally, any “amino acid tract” as referred to herein is directed to a peptide or polypeptide comprising at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty-five, at least thirty, at least thirty-five, at least forty, at least forty-five, at least fifty, at least fifty-five, at least sixty, at least sixty-five, at least seventy, at least seventy-five, at least eighty, at least eighty-five, at least ninety, at least ninety-five, or at least one-hundred consecutive identical amino acid residues, wherein said identical residues are encoded by identical codons.

A “repeat region” as used herein refers to the part of DNA where a certain codon is repeated many times. Expansions sometimes occur during replication of repeat regions. In Huntington's Disease, the repeat region involves the CAG codon. During replication, DNA polymerase III dissociates from the DNA template strand at the repeat region and the subsequent reattachment at a more distant site. Polymerase slippage can cause the newly created DNA strand to contain an expanded section of DNA.

According to an exemplary embodiment of this aspect of the present invention, the polypeptide sFlt1-14 (also denoted as SEQ ID NO. 2), encoded by the polynucleotide described herein (also denoted as SEQ ID NO. 3, or any fragments, derivatives and homologues thereof) is capable of binding a VEGFR ligand. Examples of such ligands include, without limitation, VEGF (VEGF-A, GeneBank Accession No. NP_{—}001020537), VEGF-B (GeneBank Accession No. NP_{—}003368) and Placenta growth factor (PlGF, GeneBank Accession No. NP_{—}002623).

According to another exemplary embodiment, binding of the polypeptide is expected to be in a range of about 10⁻⁹ M-10⁻¹² M.

The polynucleotide of the invention, or fragments thereof as described, may be incorporated into certain constructs. The present invention further concerns an expression construct comprising the above nucleic acid sequences and regulatory elements, and further concerns plasmids comprising the same.

Thus, in a second aspect, the present invention contemplates a nucleic acid construct comprising a nucleic acid sequence coding for sFlt1-14 or any fragment thereof comprising the serine-rich C-terminus region of sFlt1-14. At least one of the TCA serine coding codons in the serine-rich C-terminus region of sFlt1-14 as encoded by the nucleic acid sequence of SEQ ID NO. 1 is replaced by any one of TCT, TCC, TCG, AGT, AGC, and the construct optionally further comprises operably linked regulatory elements.

“Construct”, as used herein, encompasses vectors such as plasmids, viruses, bacteriophage, integratable DNA fragments, and other vehicles, which enable the integration of DNA fragments into the genome of the host.

In some embodiments, the nucleic acid construct of the invention comprises a nucleic acid sequence at least 31% homologous to the serine-rich C-terminus encoding region of SEQ ID NO. 3.

According to more specific embodiments, the nucleic acid construct of the invention comprises the nucleic acid sequence as denoted by SEQ ID NO. 3, or any fragment thereof encoding the serine-rich C-terminus region of sFlt1-14.

In another aspect, the invention discloses an expression vector comprising the nucleic acid construct of the invention.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

As used herein, the term “gene” or “recombinant gene” refers to a nucleic acid comprising an open reading frame encoding a peptide or polypeptide, including both exon and (optionally, as in the present case) intron sequences. A “recombinant gene” refers to nucleic acid encoding a peptide or polypeptide and comprising exon sequences, though it may optionally include intron sequences, wherein at least one nucleotide comprised in the nucleic acids comprising said gene was changed from the native, original sequence, using artificial means.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass. (1988), which are incorporated herein by reference.

To enable cellular expression of the polynucleotides of the present invention, the nucleic acid construct of the present invention further includes and operably linked to regulatory elements, for example, at least one cis acting regulatory element. As used herein, the phrase “cis acting regulatory element” refers to a polynucleotide sequence, preferably a promoter, which binds a trans acting regulator and regulates the transcription of a coding sequence located downstream thereto. The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Any suitable promoter sequence can be used by the nucleic acid construct of the present invention. Preferably, the promoter utilized by the nucleic acid construct of the present invention is active in the specific cell population transformed. Examples of cell type-specific and/or tissue-specific promoters include promoters such as albumin that is liver specific, lymphoid specific promoters, neuron-specific promoters such as the neurofilament promoter, pancreas-specific promoters, or mammary gland-specific promoters such as the milk whey promoter. The nucleic acid construct of the present invention can further include an enhancer, which can be adjacent or distant to the promoter sequence and can function in up regulating the transcription therefrom.

Depending on the host/vector system utilized, any of a number of suitable transcription and translation elements including constitutive and inducible promoters, transcription enhancer elements, transcription terminators, and the like, can be used in the expression vector. Other than containing the necessary elements for the transcription and translation of the engineered sequence of the invention, the expression construct of the present invention can also include sequences engineered to optimize stability, production, purification, yield or toxicity of the expressed fusion protein.

The nucleic acid construct of the present invention may further include an appropriate selectable marker and/or an origin of replication. For example, the nucleic acid construct utilized may be a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives. Expression vectors containing regulatory elements from eukaryotic viruses such as retroviruses can be also used. SV40 vectors include pSVT7 and pMT2. It should be appreciated that the present invention encompasses any vectors that were used in the present invention as described by the examples.

Vectors derived from bovine papilloma virus include pBV-IMTHA, and vectors derived from Epstein Bar virus include pHEBO, and p2O5. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Recombinant viral vectors may also be used to synthesize the polynucleotides of the present invention. Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Bone marrow cells can be targeted using the human T cell leukemia virus type I (HTLV-I).

Currently preferred in vivo nucleic acid transfer techniques include transfection with viral or non-viral constructs, such as adenovirus, lentivirus, Herpes simplex I virus, or adeno-associated virus (AAV) and lipid-based systems. Very efficient constructs for use in gene therapy are viruses, most specifically, adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al, Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and include, for example, stable or transient transfection, lipofection, electroporation and infection with recombinant viral vectors.

As used herein, the term “transfection” means the introduction of a nucleic acid, e.g., an expression vector, into a recipient cells by nucleic acid-mediated gene transfer. “Transformation”, as used herein, refers to a process in which a cell's genotype is changed as a result of the cellular uptake of exogenous DNA or RNA. Methods of introducing the expression construct into a host cell are well known in the art and include electroporation, lipofection and chemical transformation (e.g., calcium phosphate). The “transformed” cells are cultured under suitable conditions, which allow the expression of the polypeptide encoded by the nucleic acid sequence. Following a predetermined time period, the expressed molecule is recovered from the cell or cell culture, and purification is effected according to the end use of the recombinant polypeptide.

Introduction of nucleic acids by viral infection offers several advantages over other methods such as lipofection and electroporation, since higher transfection efficiency can be obtained due to the infectious nature of viruses.

In cases where large amounts of the peptides encoded by the polynucleotide of the present invention are desired, the polypeptides expressed by the method of the present invention can be generated using recombinant techniques. Briefly, an expression construct (i.e., expression vector), which includes the engineered polynucleotide of the present invention (e.g., SEQ ID NO: 3, or any variants or fragments thereof, for example, the fragments of SEQ ID NO. 15 to 18 and SEQ ID NO. 23, 25, 27, 29, 31, 33, 35, 37, 39 and 41), positioned under the transcriptional control of a regulatory element, such as a promoter (as explained in detail herein), is introduced into host cells.

Thus, a further aspect of the invention relates to a host cell transformed or transfected with the expression vector of the invention.

“Cells”, “host cells” or “recombinant cells” are terms used interchangeably herein. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein. “Host cell” as used herein refers to cells which can be recombinantly transformed or transfected with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival.

Suitable host cells include prokaryotes, lower eukaryotes, and higher eukaryotes. Prokaryotes include gram negative and gram positive organisms, e.g., E. coli and B. subtilis. Lower eukaryotes include yeast, S. cerevisiae and Pichia, and species of the genus Dictyostelium. Higher eukaryotes include established tissue culture cell lines from animal cells, both of non-mammalian origin, e.g., insect cells and birds, and of mammalian origin, e.g., human and other primate, and of rodent origin.

More specifically, a variety of prokaryotic or eukaryotic cells can be used as host-expression systems to express the engineered polynucleotide sequence of the invention. These include, but are not limited to, microorganisms, such as bacteria transformed with a recombinant bacteriophage DNA, plasmid DNA or cosmid DNA expression vector containing the engineered sequence of the invention; yeast transformed with recombinant yeast expression vectors containing the engineered sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the engineered sequence. Mammalian expression systems are preferably used to express the engineered sequence of the present invention.

The choice of host cell line for the expression of the molecules depends mainly on the expression vector. Eukaryotic expression systems are preferred (e.g., mammalian and insects) since they allow post translational modifications (e.g., glycosylation). Another consideration is the amount of protein that is required. Milligram quantities often can be produced by transient transfections. For example, the adenovirus EIA-transformed 293 human embryonic kidney cell line can be transfected transiently with pRK5-based vectors by a modification of the calcium phosphate method to allow efficient expression. CDM8-based vectors can be used to transfect COS cells by the DEAE-dextran method. If larger amounts of protein are desired, the molecules can be expressed after stable transfection of a host cell line. It will be appreciated that the presence of a hydrophobic leader sequence at the N-terminus of the molecule will ensure processing and secretion of the molecule by the transfected cells. It will be appreciated that the use of bacterial or yeast host systems may be preferable to reduce cost of production. However since bacterial host systems are devoid of protein glycosylation mechanisms, a post production glycosylation may be needed.

In any case, transformed cells are cultured under effective conditions, which allow for the expression of high amounts of recombinant polypeptide encoded by the polynucleotide of the present invention. Effective culture conditions include, but are not limited to, effective media, bioreactor, temperature, pH and oxygen conditions that permit protein production. An effective medium refers to any medium in which a cell is cultured to produce the recombinant chimera molecule of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. Depending on the vector and host system used for production, resultant proteins of the present invention may either remain within the recombinant cell, secreted into the fermentation medium, secreted into a space between two cellular membranes, such as the periplasmic space in E. coli; or retained on the outer surface of a cell or viral membrane. Following a predetermined time in culture, recovery of the recombinant protein is effected.

According to a further aspect of the present invention, a pharmaceutical composition comprising the isolated polynucleotide according to the invention or any construct, expression vector or host cell comprising the same is provided. The composition further comprises a pharmaceutically acceptable carrier.

It should be recognized that the composition of the invention may comprise any of the polynucleotides described by the invention as an active ingredient. For example, a polynucleotide comprising a nucleic acid sequence coding for sFlt1-14 or any fragment thereof comprising the serine-rich C-terminus region of said sFlt1-14, wherein at least one of the TCA serine coding codons in said serine-rich C-terminus region of sFlt1-14 as encoded by the nucleic acid sequence of SEQ ID NO. 1, is replaced by any one of TCT, TCC, TCG, AGT, AGC. In yet another embodiment, the composition of the invention may comprise a polynucleotide where the serine coding codons comprised within the serine-rich C-terminus encoding sequence thereof are either followed, preceded or both by a non-identical codon.

In particular embodiments, the composition of the invention may comprises a polynucleotide, wherein at least one codon of each at least two successive serine coding codons comprised within the serine-rich C-terminus encoding sequence of the polynucleotide of the invention are either followed, preceded or both by a non-identical serine codon selected from the group consisting of TCA, TCT, TCC, TCG, AGT, AGC.

In more specific embodiments, each codon of each at least two successive serine coding codons comprised within the serine-rich C-terminus encoding sequence of the polynucleotide comprised within the composition of the invention, are either followed, preceded or both by a non-identical serine codon selected from the group consisting of TCA, TCT, TCC, TCG, AGT, AGC.

In yet more specific embodiments, each codon of at least two successive serine coding codons selected from the group consisting of TCA, TCT, TCC and TCG comprised within the serine-rich C-terminus encoding sequence of the polynucleotide comprised within the composition of the invention are either followed, preceded or both by a non-identical serine codon selected from the group consisting of AGT and AGC.

In one specific embodiment, the polynucleotide comprised within the composition of the invention may comprise a nucleic acid sequence at least 31% homologous to the serine-rich C-terminus encoding region of SEQ ID NO. 3.

Specific embodiments of the invention contemplate the pharmaceutical composition according of the invention, wherein the polynucleotide comprises the nucleic acid sequence as denoted by SEQ ID NO. 3 or any fragment thereof encoding the serine-rich C-terminus region of sFlt1-14. Non-limiting examples for polynucleotides encoding fragments of the serine-rich C-terminus region of sFlt1-14 are denoted by any one of SEQ ID NO. 15 to 18 and SEQ ID NO. 23, 25, 27, 29, 31, 33, 35, 37, 39 and 41.

In another embodiment, the invention provides a pharmaceutical composition according to the invention, for the treatment of a VEGF-associated medical condition.

It should be noted that formulations used by the compositions and methods of the invention include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

As indicated above, pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention, i.e., the polynucleotide or any fragment or construct thereof, may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

Pharmaceutical compositions for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol, cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). Thickeners, flavoring agents, diluents, emulsifiers, dispersing aids or binders may be desirable.

For administration by nasal inhalation, the active ingredient for use according to the present invention, which is the polynucleotide, construct or vector of the invention, or preferably, a pharmaceutical composition comprising the same, may conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Thus, the pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

As indicated above, determination of a therapeutically effective amount is well within the capability of those-skilled in the art. For any pharmaceutical composition used by the treatment method of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from in vitro cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition.

Depending on the severity and responsiveness of the condition to be treated, dosing can be of a single or a plurality of administrations, with course of treatment lasting from several days to several weeks or until cure is effected or diminution of the disease state or symptoms is achieved.

The amount of the pharmaceutical composition to be administered will of course be dependent on the subject being treated, the severity of the affliction, the manner of administration, the judgment of the prescribing physician, etc.

As previously disclosed in WO2008075363, the present inventors have revealed, for the first time, that sFlt1-14 (and not sFlt-1), which specifically binds and antagonizes circulating VEGF and PlGF, is the soluble receptor found in the serum of preeclamptic subjects. Thus, sFlt1-14 is the major VEGF receptor in the circulation of preeclamptic subjects, and as such was suggested as a marker of and target for treating this condition. This variant is soluble, secreted, comprises a unique amino acid sequence (SEQ ID NO: 2) and is expressed during preeclampsia.

As disclosed in WO2008075363, the sFlt1-14 receptor is expressed in placentae and is highly upregulated in preeclamptic placentae and provides a valuable indicator of preeclampsia or predisposition thereof. Furthermore, since sFlt1-14 functions in antagonizing VEGFR ligands (e.g., VEGF), modulating sFlt1-14 levels (e.g. downregulating or upregulating) may serve as a powerful tool in treatment of VEGF associated conditions.

Thus, according to a further aspect, the invention discloses a method for the treatment of a VEGF-associated medical condition comprising the step of administering to a subject in need thereof a therapeutically effective amount of the isolated polynucleotide according to the invention, or any construct, expression vector, host cell or composition comprising the same.

VEGF associated conditions may include for example, hyper angiogenesis, cancer and neovascularized cornea. As used herein the term “angiogenesis” refers to the production or development of blood vessels. As used herein the term “cancer” refers to any tumoral disease including metastasis. Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Endometrial cancer, Malignant melanoma, Mesothelioma, breast, skin, prostate, and ovarian. As used herein the phrase “neovascularized cornea” refers to the abnormal, pathological condition in which the cornea becomes vascular.

Other medical conditions, diseases and disease processes in which angiogenesis plays a role can be treated using the polypeptide expressed using the polynucleotide of the invention according to the method of the present invention.

The method of the invention may be applicable for treating a subject suffering from a VEGF-associated disorder. As used herein, the term “disorder” refers to a condition in which there is a disturbance of normal functioning. A “disease” is any abnormal condition of the body or mind that causes discomfort, dysfunction, or distress to the person affected or those in contact with the person. Sometimes the term is used broadly to include injuries, disabilities, syndromes, symptoms, deviant behaviors, and atypical variations of structure and function, while in other contexts these may be considered distinguishable categories. It should be noted that the terms “disease”, “disorder”, “condition” and “illness”, are equally used herein.

The terms “treat, treating, treatment” as used herein and in the claims mean ameliorating one or more clinical indicia of disease activity in a patient having a pathologic disorder.

“Treatment” refers to therapeutic treatment. Those in need of treatment are mammalian subjects suffering from any VEGF-associated disorder. By “patient” or “subject in need” is meant any mammal for which administration of the polynucleotides, constructs or vectors of the invention, or any pharmaceutical composition of the invention is desired, in order to prevent, overcome or slow down such infliction.

The terms “effective amount” or “sufficient amount” as used by the methods of the invention, mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

Although the method of the invention is particularly intended for the treatment of pathologic disorders in humans, other mammals are included. By way of non-limiting examples, mammalian subjects include monkeys, equines, cattle, canines, felines, mice, rats and pigs.

It will be appreciated that expression of other peptides or polypeptides comprising identical amino-acid tracts encoded by identical triplets, wherein said amino acid may be encoded by at least two different codons, are also envisaged by the present invention.

In particular embodiments, the present invention discloses methods of treatment of subjects suffering from VEGF-associated disorders, by introducing to said subjects the polynucleotide of the invention, or fragments thereof according to the invention. This may be accomplished by gene therapy and gene delivery methods known in the art. The introduction of said polynucleotide may be into germline (eggs or sperm) or somatic (most cells of the body) cells of the subject. In theory, it is possible to transform either somatic cells or germ cells. Gene therapy using germ line cells results in permanent changes that are passed down to subsequent generations. If done early in embryologic development, such as during preimplantation diagnosis and in vitro fertilization, the gene transfer could also occur in all cells of the developing embryo. The appeal of germ line gene therapy is its potential for offering a permanent therapeutic effect for all who inherit the target gene. Successful germ line therapies introduce the possibility of eliminating some diseases from a particular family, and ultimately from the population, forever.

Somatic cells are nonreproductive. Somatic cell therapy is viewed as a more conservative, safer approach because it affects only the targeted cells in the patient, and is not passed on to future generations. In other words, the therapeutic effect ends with the individual who receives the therapy. However, this type of therapy presents unique problems of its own. Often the effects of somatic cell therapy are short-lived. Because the cells of most tissues ultimately die and are replaced by new cells, repeated treatments over the course of the individual's life span are required to maintain the therapeutic effect. Transporting the gene to the target cells or tissue is also problematic. Regardless of these difficulties, however, somatic cell gene therapy is appropriate and acceptable for many disorders. Clinicians can even perform this therapy in utero, potentially correcting or treating a life-threatening disorder that may significantly impair a baby's health or development if not treated before birth.

The methods of treatment of the invention also include the use of gene delivery systems to introduce the polynucleotide of the invention, or fragments thereof, into subjects in need thereof. Various procedures and instruments used in said gene delivery are known to the man of the art. For example, such instruments and methods comprise the gene gun, the impalefection procedure and the gene electrotransfer method.

The gene gun is part of a method called the biolistic (also known as bioballistic) method, and under certain conditions, DNA (or RNA) become “sticky,” adhering to biologically inert particles such as metal atoms (usually tungsten or gold). By accelerating this DNA-particle complex in a partial vacuum and placing the target tissue within the acceleration path, DNA is effectively introduced. Uncoated metal particles could also be shot through a solution containing DNA surrounding the cell thus picking up the genetic material and proceeding into the living cell. A perforated plate stops the shell cartridge but allows the slivers of metal to pass through and into the living cells on the other side. The cells that take up the desired DNA, identified through the use of a marker gene (in plants the use of GUS is most common), are then cultured to replicate the gene and possibly cloned. The biolistic method is most useful for inserting genes into plant cells such as pesticide or herbicide resistance. Different methods have been used to accelerate the particles: these include pneumatic devices; instruments utilizing a mechanical impulse or macroprojectile; centripetal, magnetic or electrostatic forces; spray or vaccination guns; and apparatus based on acceleration by shock wave, such as electric discharge. There are several variables in these experiments that must be controlled in order to attain maximal transformation efficiency. Optimal responses have been shown to be dependent on the delivery of a sufficient number of DNA-coated particles, as well as how much DNA coats the metal particles. Temperature, amount of cells, and their ability to regenerate also has an effect on the overall efficiency, as well as the type of gun used: helium powered vs. gun-powder, hand-held vs. stand-alone, etc. It is also important to adjust the length of the flight path of the particles: fragile tissues cannot be bombarded at the same high speed as those which have more resistance to foreign particles entering. How to adjust these variables depends mainly on which metal particles one uses to transfer the genetic material, and what type of cells one is attempting to transfect.

Impalefection is a method of gene delivery using nanomaterials, such as carbon nanofibers, carbon nanotubes and nanowires. Needle-like nanostructures are synthesized perpendicular to the surface of a substrate. Plasmid DNA containing the gene, intended for intracellular delivery, is attached to the nanostructure surface. A chip with arrays of these needles is then pressed against cells or tissue. Cells that are impaled by nanostructures can express the delivered gene(s). As one of the types of transfection, the term is derived from two words—impalement and infection. Vertically aligned carbon nanofiber arrays prepared by photolithography and plasma enhanced chemical vapor deposition are one of the suitable types of material. Silicon nanowires were also used as nanoneedles in impalefection.

The electrotransfer method comprises the application of electric pulses of sufficient strength to the cell, causing an increase in the trans-membrane potential difference, which provokes the membrane destabilization. Cell membrane permeability is increased and otherwise nonpermeant molecules enter the cell. Although the mechanisms of gene electrotransfer are not yet fully understood, it was shown that the introduction of DNA only occurs in the part of the membrane facing the cathode and that several steps are needed for successful transfection: electrophoretic migration of DNA towards the cell, DNA insertion into the membrane, translocation across the membrane, migration of DNA towards the nucleus, transfer of DNA across the nuclear envelope and finally gene expression. There are a number of factors that can influence the efficiency of gene electrotransfer, such as: temperature, parameters of electric pulses, DNA concentration, electroporation buffer used, cell size and the ability of cells to express transfected genes. In in vivo gene electrotransfer, also DNA diffusion through extracellular matrix, properties of tissue and overall tissue conductivity are crucial. Gene electrotransfer became of special interest because of its low cost, easiness of realization and safety. Namely, viral vectors can have serious limitations in terms of immunogenicity and pathogenicity when used for DNA transfer. Although the method is not systemic, but strictly local one, it is still the most efficient non-viral strategy for gene delivery.

In a specific embodiment, the method of the present invention is also directed to the modification of nucleic acid sequences comprising repeat regions; fragments thereof, or sequences encoding similar polypeptides with different but successive identical codon usage, “successive” meaning that at least two consecutive identical codons are present within said nucleic acid sequence, wherein said codons encode an amino acid that may be encoded by more than a single codon sequence, thus permitting appropriate replication of constructs comprising said sequence and, subsequently, expression of the peptide or polypeptide encoded by said sequence.

In a further aspect, the invention contemplates a method for efficient propagation and expression of a nucleic acid sequence coding for sFlt1-14 or any fragment thereof comprising the serine-rich C-terminus region of sFlt1-14. The method comprises the step of providing a polynucleotide sequence encoding said sFlt1-14 or any construct or expression vector thereof, wherein at least one of the TCA serine coding codons in the serine-rich C-terminus region of sFlt1-14 as encoded by the nucleic acid sequence of SEQ ID NO. 1 is replaced by any one of TCT, TCC, TCG, AGT, and AGC.

In one embodiment of the method of the invention, at least thirteen of the serine coding codons comprised within the serine-rich C-terminus encoding sequence of the polynucleotide of the invention is either followed, preceded or both by a non-identical codon.

In another embodiment of the method of the invention, all serine coding codons comprised within the serine-rich C-terminus encoding sequence of the polynucleotide of the invention are either followed, preceded or both by a non-identical codon.

In yet another embodiment, the method of the invention provides the use of an isolated polynucleotide having no more than five identical serine coding codons in tandem.

In particular embodiments of the method of the invention, at least one codon of each of at least two successive serine coding codons comprised within the serine-rich C-terminus encoding sequence of the polynucleotide of the invention are either followed, preceded or both by a non-identical serine codon selected from the group consisting of TCA, TCT, TCC, TCG, AGT, AGC.

In other embodiments of the method of the invention, each codon of each at least two successive serine coding codons comprised within the serine-rich C-terminus encoding sequence of the polynucleotide of the invention are either followed, preceded or both by a non-identical serine codon selected from the group consisting of TCA, TCT, TCC, TCG, AGT, AGC.

In specific embodiments of the method of the invention, each codon of each at least two successive serine coding codons selected from the group consisting of TCA, TCT, TCC and TCG comprised within the serine-rich C-terminus encoding sequence of the polynucleotide of the invention are either followed, preceded or both by a non-identical serine codon selected from the group consisting of AGT and AGC.

In yet other embodiments of the method of the invention, the polynucleotide of the invention comprises a nucleic acid sequence at least 31% homologous to the serine-rich C-terminus encoding region of SEQ ID NO. 3.

In specifically preferred embodiments of the method of the invention, the polynucleotide of the invention comprises the nucleic acid sequence as denoted by SEQ ID NO. 3 or any fragment thereof encoding the serine-rich C-terminus region of sFlt1-14. Non-limiting examples for polynucleotides encoding fragments of the serine-rich C-terminus region of sFlt1-14 are denoted by any one of SEQ ID NO. 15 to 18 and SEQ ID NO. 23, 25, 27, 29, 31, 33, 35, 37, 39 and 41.

The method of the invention facilitates the encoding polynucleotide propagation and expression of polyserine tracts, such as the polyserine tract comprised in the Flt-1 soluble splice variant sFlt1-14. As used herein the term “soluble” refers to the ability of the molecules of the present invention to dissolve in a physiological aqueous solution (pH about 7, e.g., solubility level in aqueous media of, at least, 100 μg/ml) without substantial aggregation. Thus, it is readily understood that soluble sFlt1-14 are preferably devoid of hydrophobic transmembrane domains. Being soluble, the polypeptides of the present invention may be secreted.

Thus, in specific embodiments, the present invention concerns a method of modification of a nucleic acid sequence coding for the at least the serine-rich C-terminal region of sFlt1-14, wherein at least 5% of the TCA serine coding codons which appear in the native sequence as depicted in FIG. 1 and denoted by SEQ ID NO. 1, are replaced by non-TCA serine coding codons.

As described earlier, expression of the splice variant sFlt1-14 (denoted by SEQ ID NO.:2) is hampered by interference with plasmid replication, likely as a result of polymerase slippage occurring due to the C′-terminal polyserine encoded by the C′-terminal sFlt1-14 in intron 14. Since sFlt1-14 may serve both as a diagnostic marker, and as a therapeutic compound for the treatment of VEGF associated conditions, the expression of either the full sFlt1-14 or, at the very least, the serine-rich C-terminus region of sFlt1-14, is sought.

The “at least 5% of the TCA codons coding for serine are replaced by non-TCA serine coding codons”, means that some of the naturally appearing codons for serine, that are mostly TCA, are replaced with the other five possible codons of serine within the degeneracy of the genetic code, so that there is no long stretch of repeated triplets, made up of either the natural TCA or the replacing codons, in tandem. In order to avoid slippage, it is best that no identical codons are present in tandem. It is possible to use one, two, three, for or all five of non-TCA codons (TCT, TCC, TCG, AGT, AGC), and mix several codon as long as there is no long stretch of identical tandem codon repeats. TCA can still be used as long as there are no adjacent TCA codons next to it.

The method of the invention overcomes the interference with nucleic acid replication (and expression) caused by regions of repeating identical codons, i.e. stretches of at least two identical codons encoding amino acids which may be encoded by at least two different codons. Thus, the inventors show that alteration of nucleotides within said repeat region permits proper replication and expression of said polynucleotide. The polynucleotide coding region may be engineered by taking advantage of the degeneracy of the genetic code to alter the coding sequence such that, while the nucleotide sequence is substantially altered, it nevertheless encodes a protein having an amino acid sequence identical to the native protein or peptide. Based upon the degeneracy of the genetic code, variant DNA molecules may be derived from the cDNA and gene sequences using standard DNA mutagenesis techniques or by synthesis of DNA sequences. The term “within the degeneracy of the genetic code” used herein means possible usage of any nucleotide combinations as codons that code for the same amino acid. In other words, such changes in the nucleic acid sequences that are not reflected in the amino acid sequence of the encoded protein.

Preferably the non-TCA occurring codons are distributed so that no more than 5, preferably no more than 4, more preferably no more than 3, most preferably no more than 2 identical serine coding codons are present in tandem.

As stated hereinabove, the method of the invention also encompasses the encoding polynucleotide propagation and expression of fragments (e.g., as short as a specific antigenic determinant e.g., at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 39 amino acids such as derived from SEQ ID NO: 2, or any fragment thereof) of the above described polynucleotides and polypeptides. These fragments may be used to elicit antibody production against the isolated polypeptides of the invention.

Antibodies raised against the isolated polypeptides of the invention may be used for diagnostic purposes on biological samples, e.g. for diagnosis of preeclamptic subjects by detection of sFlt1-14 in their serum.

Specific peptides chosen for antibody generation are preferably selected immunogenic (i.e., capable of stimulating an antibody response). Parameters for testing peptide immunogenicity are well known in the art including, but not limited to, molecular size, chemical composition and heterogeneity and susceptibility to antigen processing and presentation. Non-limiting examples of serine-rich C′-terminal sFlt1-14 fragments that may be useful for the invention, for example, for use as epitopes in creating antibodies, comprise amino acid sequence as denoted by SEQ ID NO.: 24 and encoded by SEQ ID NO.: 23, amino acid sequence as denoted by SEQ ID NO.: 26 and encoded by SEQ ID NO.: 25, amino acid sequence as denoted by SEQ ID NO.: 28 and encoded by SEQ ID NO.: 27, amino acid sequence as denoted by SEQ ID NO.: 30 and encoded by SEQ ID NO.: 29, amino acid sequence as denoted by SEQ ID NO.: 32 and encoded by SEQ ID NO.: 31, amino acid sequence as denoted by SEQ ID NO.: 34 and encoded by SEQ ID NO.: 33, amino acid sequence as denoted by SEQ ID NO.: 36 and encoded by SEQ ID NO.: 35, amino acid sequence as denoted by SEQ ID NO.: 38 and encoded by SEQ ID NO.: 37, amino acid sequence as denoted by SEQ ID NO.: 40 and encoded by SEQ ID NO.: 39 and amino acid sequence as denoted by SEQ ID NO.: 42 and encoded by SEQ ID NO.: 41.

It will be appreciated that the above described antibodies for detection of sFlt1-14 is mostly desired for the diagnosis of a pregnancy associated medical condition associated with maternal or fetal stress. As used herein the term “diagnosis” refers to classifying a disease or a symptom, determining a severity of such a disease, monitoring disease progression, monitoring the effectiveness of a therapeutic regime, forecasting (prognosing) an outcome of a disease and/or prospects of recovery. As used herein the phrase “a pregnancy associated medical condition associated with maternal or fetal stress” refers to a disease or a syndrome in which there are clinical symptoms in the mother of fetus which are associated with upregulation of sFlt1-14. The pregnancy may be at any stage or phase. The medical condition may include any hypertensive disorders: preeclampsia, eclampsia, mild preeclampsia, chronic hypertension, EPH gestosis, gestational hypertension, superimposed preeclampsia (including preeclampsia superimposed on chronic hypertension, chronic nephropathy or lupus), HELLP syndrome (hemolysis, elevated liver enzymes, low platelet count) or nephropathy. The medical condition may also include gestational diabetes, fetal growth restriction (FGR) and fetal alcohol syndrome (FAS). As used herein, the phrase “maternal or fetal stress” refers to any condition in which the mother or the fetus is at risk of developing a pregnancy related complication. Fetal stress includes, without being limited to, inadequate nutrient supply and cessation of fetal growth. Maternal stress includes, without being limited to, hypertension and diabetes. Fetal and maternal stress may affect fetal development and brain functions and plays a significant role in pregnancy outcomes related to prematurity and urgent deliveries (e.g. c-section).

It should not be overlooked that by demonstrating an efficient propagation and expression of the engineered polynucleotide described herein, the invention further exemplifies the feasibility of using differential codon usage for efficient propagation and expression of other proteins containing tract of identical amino acids. Therefore, the invention further provides a method for efficient propagation and expression of a nucleic acid sequence encoding a protein of interest wherein said protein comprises a tract of identical amino acids, said amino acid residues are encoded by at least two different codons, said method comprises the step of providing a polynucleotide sequence encoding said protein of interest wherein each of the codons coding for said identical amino acids is at least one of followed and preceded by a non-identical codon or a non identical codon encoding said amino acid.

As used herein the term “about” refers to ±10% The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Reference is now made to the following examples, which together with the above descriptions, illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley; Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998). “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. L, ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Experimental Procedures

### Sequences

Table 2 summarizes all sequences disclosed in the present specification.

TABLE 2
Sequences SEQ ID NO: 1-43

| SEQ ID NO. | Sequence name |
|---|---|
| 1 | sFlt1-14 nucleic acid sequence |
| 2 | sFlt1-14 protein |
| 3 | Engineered sFlt1-14 |
| 4 | Intron 14 amino acid sequence |
| 5 | Intron 14 nucleic acid sequence |
| 6 | Engineered intron 14 nucleic acid sequence |
| 7 | sFlt1-14 serine-rich region (i) amino acid sequence |
| 8 | sFlt1-14 serine-rich region (ii) amino acid sequence |
| 9 | sFlt1-14 serine-rich region (iii) amino acid sequence |
| 10 | sFlt1-14 serine-rich region (iv) amino acid sequence |
| 11 | sFlt1-14 serine-rich region (i) nucleic acid sequence |
| 12 | sFlt1-14 serine-rich region (ii) nucleic acid sequence |
| 13 | sFlt1-14 serine-rich region (iii) nucleic acid sequence |
| 14 | sFlt1-14 serine-rich region (iv) nucleic acid sequence |
| 15 | Engineered sFlt1-14 serine-rich region (v) nucleic acid |
| | sequence |
| 16 | Engineered sFlt1-14 serine-rich region (vi) nucleic acid |
| | sequence |
| 17 | Engineered sFlt1-14 serine-rich region (vii) nucleic acid |
| | sequence |
| 18 | Engineered sFlt1-14 serine-rich region (viii) nucleic acid |
| | sequence |
| 19 | Forward primer |
| 20 | Reverse primer |
| 21 | CESS epitope |
| 22 | sFlt1 amino acid sequence |
| 23 | Fragment 1 nucleic acid sequence |
| 24 | Fragment 1 amino acid sequence |
| 25 | Fragment 2 nucleic acid sequence |
| 26 | Fragment 2 ammo acid sequence |
| 27 | Fragment 3 nucleic acid sequence |
| 28 | Fragment 3 amino acid sequence |
| 29 | Fragment 4 nucleic acid sequence |
| 30 | Fragment 4 amino acid sequence |
| 31 | Fragment 5 nucleic acid sequence |
| 32 | Fragment 5 amino acid sequence |
| 33 | Fragment 6 nucleic acid sequence |
| 34 | Fragment 6 amino acid sequence |
| 35 | Fragment 7 nucleic acid sequence |
| 36 | Fragment 7 amino acid sequence |
| 37 | Fragment 8 nucleic acid sequence |
| 38 | Fragment 8 amino acid sequence |
| 39 | Fragment 9 nucleic acid sequence |
| 40 | Fragment 9 amino acid sequence |
| 41 | Fragment 10 nucleic acid sequence |
| 42 | Fragment 10 amino acid sequence |
| 43 | Reverse primer for sFlt1-14 amplification |

### Antibodies

*Ab9540, (Abcam)—monoclonal mouse anti-human VEGF Receptor 1 antibody directed against the extracellular domain of both sFlt-1 and sFlt-14, raised using as an immunogen recombinant human soluble extracellular Flt-11 g-like loop 1 to 5 (sFlt-1(D5)), Cat No. Ab9540.
*CESS—polyclonal rabbit antibody directed against a peptide derived from the C-terminus of the sFLT1-14 protein—CELYTSTSPSSSSSS (denoted as SEQ ID NO.: 21), as described in WO2008075363.

### Cell Culture

*Hela cells

ATCC number: CCL-2, the base medium for this cell line is ATCC-formulated Eagle's Minimum Essential Medium, Catalog No. 30-2003. To make the complete growth medium, add the following components to the base medium: fetal bovine serum to a final concentration of 10%.

Cloning of the Engineered sFlt1-14

Natural sFlt1-14 was used as a template for the PCR amplification of engineered sFlt1-14 (also denoted as SEQ ID NO.: 1, and 3, respectively). The primers used for the amplification were: Forward Primer: atggtcagctactgggacac (also denoted as SEQ ID NO.: 19) and Reverse Primer: ctatgaactagagctggaacttgagctagaactggagctcaatggagagcttgacgatgacgatg gtgacg (also denoted as SEQ ID NO.: 20).

The PCR fragment of the amplified engineered sFlt1-14 was cloned into a pCR2.1-TOPO vector, and transfected to one shot TOP10 chemically competent E. coli. Transformed colonies were grown in 200 ml LB and plasmid was produced with the HiSpeed plasmid maxi kit (QIAGEN #12662). Plasmid production yield was determined using Nanodrop 2000c (Thermo scientific).

Isolation of sFlt1-14

Human placenta was homogenated and RNA was extracted using TRI reagent (Sigma, cat. No. T9424). cDNA was prepared with the VERSO cDNA kit (Thermo scientific, cat No. AB-1453/B). sFlt1-14 coding region was PCR amplified using the Forward primer: atggtcagctactgggacac (as denoted by SEQ ID NO. 19) and the Reverse primer: cttggctctccaactaaagg (as denoted by SEQ ID NO. 43).

### Transfection

The PCR-amplified engineered sFlt1-14 was cloned into the pBluescript vector, and was transfected to HeLa cells using DOTAP liposomal tranfection reagent (Cat. No. 1811177, Roche) Immediately prior to transfection, the cells were infected with a vaccinia virus expressing T7 polymerase to allow expression from the pBluscript vector. The cells were harvested 24 hours after transfection. sFlt1-14 was confirmed in medium as well as in the cells by ELISA (Cat. No. DVR100B, R&D systems), and cellular expression was further confirmed by western blot.

### Western Blot

Cellular proteins from transfected HeLa cells were separated on 6% acrylamide gel run electrophoreticaly, transferred to a membrane, reacted with the CESS or Ab9540 antibody, as indicated, and developed using Super-Signal® kit for peroxidase-conjugated antibody detection (Pierce Inc., Rockford).

### Example 1

### Modification of the Poly-Serine-Encoding Repeat Region Enhanced Plasmid Propagation

As stated earlier, the inventors had previously described a splice variant of the Flt1, being soluble and expressing a segment from Flt1's intron 14 (as well as exon 14). The inventors have tried to produce the plasmid coding for sFlt1-14 of WO2008075363 (the native sequence of which is shown in FIG. 1A-1B and also denoted as SEQ ID NO.: 1) in bacteria and failed to produce any plasmid using the native sequence.

Since the inventors postulated that the reason for the failure was replication slippage in bacteria caused by the TCA tandem repeats coding for the poly-serine tract at the C′ terminus of the natural sFlt1-14 (the sequence of which is shown in FIG. 1 and also denoted as SEQ ID NO.: 1), an attempt was made to clone sFlt1-14 while engineering the sFlt1-14 C′ terminal serine repeat region to comprise alternating serine-encoding codons rather than identical ones.

As described in the Experimental Procedures section above, primers were used to amplify the natural sFlt1-14 while replacing the native C′-terminal TCATCACCATTGTCATCATCATCATCATCGTCATCATCATCATCATCATAG (SEQ ID NO.13) sequence with: AGCTCTCCATTGAGCTCCAGTTCTAGCTCAAGTTCCAGCTCTAGTTCATAG, (SEQ ID NO.9) retaining the endogenous amino-acid sequence encoded by the nucleic acid sequence (replaced nucleotides are underlined).

The amplified engineered as well as the native sFlt1-14 were cloned into a pCR2.1-TOPO vector, and transfected to one shot TOP10 chemically competent E. coli. Transformed colonies were grown and plasmid was produced using the HiSpeed plasmid maxi kit. Plasmid production yield was determined using Nanodrop. Table 3 below shows that only the engineered sFlt1-14-encoding plasmid propagated, whereas the endogenous sFlt1-14-encoding plasmids could not be harvested from the transfected bacteria.

TABLE 3
Engineered and endogenous sFlt1-14 encoding plasmid yield

| | |
|---|---|
| Plasmid yield | Coding plasmid |

| | | |
|---|---|---|
| 0 | μg/nl | Endogenos sFlt1-14 |
| 1-1.5 | μg/nl | Engineered sFlt1-14 of the invention with |
| | | tandem TCA codons replaced |

These results clearly show that replacement of the repetitive serine coding codons with different serine codons overcomes replication deficiencies of plasmids containing the native sequence and demonstrate the feasibility of the method of the invention as an effective tool for propagation of plasmids containing nucleic acid sequences encoding tracts of identical successive amino acid residues.

### Example 2

The Engineered sFlt1-14 Expression Construct is Capable of Generating sFlt1-14 Protein

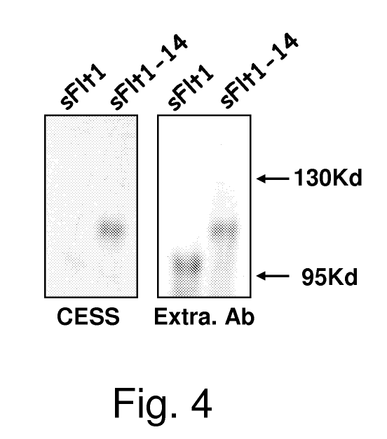

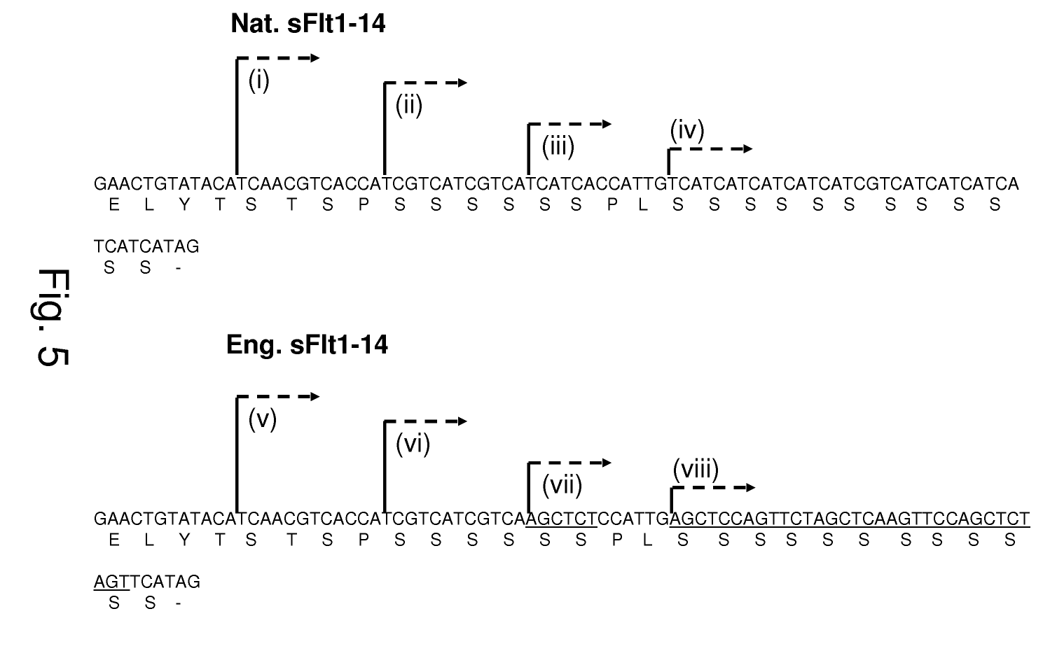

sFlt1-14 (SEQ ID NO.: 3) as well as sFlt1 (SEQ ID NO.: 22) constructs were transected to HeLa cells. Twenty-four hours later, the cells were harvested and blotted. Expression of both proteins was verified using ab9540, an antibody targeting the extracellular region common to both protein isoforms. The CESS antibody, targeting the unique C′ terminus of sFlt1-14, which contains the poly-serine tract of sFLT1-14, was used to prove that the construct is capable of producing the complete sFlt1-14. As can be seen in FIG. 4, HeLa transfected with the full sFlt-1 expressed a protein recognized by ab9540, but not by CESS. In contrast, HeLa transfected with the engineered sFLT1-14 were recognized by both antibodies, proving the correct expression of the sFLT1-14.

### Example 3

Serine-Rich C′-Terminus sFlt1-14 Fragments Expressed Using the Method of the Invention

The inventors use the method of the invention, i.e., replacement of identical serine codons in tandem, to express different fragments of the serine-rich C′-terminus. The codon replacements allow the proper propagation and expression of C′-terminus fragments comprised of amino acid sequence as denoted by SEQ ID NO.: 24 and encoded by SEQ ID NO.: 23, amino acid sequence as denoted by SEQ ID NO.: 26 and encoded by SEQ ID NO.: 25, amino acid sequence as denoted by SEQ ID NO.: 28 and encoded by SEQ ID NO.: 27, amino acid sequence as denoted by SEQ ID NO.: 30 and encoded by SEQ ID NO.: 29, amino acid sequence as denoted by SEQ ID NO.: 32 and encoded by SEQ ID NO.: 31, amino acid sequence as denoted by SEQ ID NO.: 34 and encoded by SEQ ID NO.: 33, amino acid sequence as denoted by SEQ ID NO.: 36 and encoded by SEQ ID NO.: 35, amino acid sequence as denoted by SEQ ID NO.: 38 and encoded by SEQ ID NO.: 37, amino acid sequence as denoted by SEQ ID NO.: 40 and encoded by SEQ ID NO.: 39 and amino acid sequence as denoted by SEQ ID NO.: 42 and encoded by SEQ ID NO.: 41. The engineered sFlt1-14 fragments are cloned into a pCR2.1-TOPO vector, and transfected to one shot TOP10 chemically competent E. coli. Transformed colonies are grown and plasmid is produced using the HiSpeed plasmid maxi kit.

1. An isolated polynucleotide comprising a nucleic acid sequence coding for sFlt1-14 or any fragment thereof comprising the serine-rich C-terminus region of said sFlt1-14, wherein at least one of the TCA serine coding codons in said serine-rich C-terminus region of sFlt1-14 as encoded by the nucleic acid sequence of SEQ ID NO. 1, is replaced by any one of TCT, TCC, TCG, AGT, AGC.

2. The polynucleotide according to claim 1, wherein at least thirteen of said serine coding codons comprised within said serine-rich C-terminus encoding sequence is at least one of followed and preceded by a non-identical codon.

3. An isolated polynucleotide according to claim 1 having no more than five identical serine coding codons in tandem.

4. An isolated polynucleotide according to claim 1, wherein at least 3 of the TCA serine coding codons in said serine-rich C-terminus region of sFlt1-14 as encoded by the nucleic acid sequence of SEQ ID NO. 1, are replaced by any one of TCT, TCC, TCG, AGT, AGC.

5. The polynucleotide according to claim 1, wherein at least one codon of each at least two successive serine coding codons comprised within said serine-rich C-terminus encoding sequence, is at least one of followed and preceded, by a non-identical serine codon selected from the group consisting of TCA, TCT, TCC, TCG, AGT, AGC.

6. The polynucleotide according to claim 5, wherein each codon, of each at least two successive serine coding codons comprised within said serine-rich C-terminus encoding sequence, is at least one of followed and preceded, by a non-identical serine codon selected from the group consisting of TCA, TCT, TCC, TCG, AGT, AGC.

7. The polynucleotide according to claim 6, wherein each codon of at least two successive serine coding codons selected from the group consisting of TCA, TCT, TCC and TCG is at least one of followed and preceded, by a non-identical serine codon selected from the group consisting of AGT and AGC.

8. The polynucleotide according to claim 1, wherein said sequence comprises a nucleic acid sequence at least 31% homologous to the serine-rich C-terminus encoding region of SEQ ID NO. 3.

9. The isolated polynucleotide according to claim 8, wherein said sequence comprises the nucleic acid sequence as denoted by SEQ ID NO. 3 or any fragment thereof encoding the serine-rich C-terminus region of said sFlt1-14.

10. A nucleic acid construct comprising a nucleic acid sequence coding for sFlt1-14 or any fragment thereof comprising the serine-rich C-terminus region of said sFlt1-14, wherein at least one of the TCA serine coding codons in said serine-rich C-terminus region of sFlt1-14 as encoded by the nucleic acid sequence of SEQ ID NO. 1 is replaced by any one of TCT, TCC, TCG, AGT, AGC, which construct optionally further comprises operably linked regulatory elements.

11. The nucleic acid construct according to claim 10, wherein said sequence comprises a nucleic acid sequence at least 31% homologous to the serine-rich C-terminus encoding region of SEQ ID NO. 3.

12. The nucleic acid construct according to claim 11, wherein said sequence comprises the nucleic acid sequence as denoted by SEQ ID NO. 3 or any fragment thereof encoding the serine-rich C-terminus region of said sFlt1-14.

13. An expression vector comprising the nucleic acid construct according to claim 10.

14. A host cell transformed or transfected with the expression vector according to claim 13.

15. A pharmaceutical composition comprising the isolated polynucleotide according to claim 1, or any construct, expression vector or host cell comprising the same, said composition further comprises a pharmaceutically acceptable carrier.

16. The pharmaceutical composition according to claim 15, wherein said polynucleotide comprises the nucleic acid sequence as denoted by SEQ ID NO. 3 or any fragment thereof encoding the serine-rich C-terminus region of said sFlt1-14.

17. A pharmaceutical composition according to claim 15, for the treatment of a VEGF-associated medical condition.

18. A method for the treatment of a VEGF-associated medical condition comprising the step of administering to a subject in need thereof a therapeutically effective amount of the isolated polynucleotide according to claim 1, or any construct, expression vector, host cell or composition comprising the same.

19. A method for efficient propagation and expression of a nucleic acid sequence coding for sFlt1-14 or any fragment thereof comprising the serine-rich C-terminus region of said sFlt1-14, said method comprises the step of providing a polynucleotide sequence encoding said sFlt1-14 or any construct or expression vector thereof, wherein at least one of the TCA serine coding codons in said serine-rich C-terminus region of sFlt1-14 as encoded by the nucleic acid sequence of SEQ ID NO. 1 is replaced by any one of TCT, TCC, TCG, AGT, AGC.

20. The method according to claim 19, wherein said polynucleotide sequence comprises a nucleic acid sequence at least 31% homologous to the serine-rich C-terminus encoding region of SEQ ID NO. 3.

21. The method according to claim 20, wherein said polynucleotide comprises the nucleic acid sequence as denoted by SEQ ID NO. 3 or any fragment thereof encoding the serine-rich C-terminus region of said sFlt1-14.


SEQUENCE LISTING (43 sequences)

SEQ ID NO: 1 - DNA, 2202 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctgaactgtatacatcaacgtcaccatcgtcatcgtcatcatcaccattgtcatcatcatcatcatcgtcatcatcatcatcatcatag

SEQ ID NO: 2 - protein, 733 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPELYTSTSPSSSSSSPLSSSSSSSSSSSS

SEQ ID NO: 3 - DNA, 2202 bp, Artificial
  Replaced polyserine-encoding C`-terminal region
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagagatcaggaagcaccatacctcctgcgaaacctcagtgatcacacagtggccatcagcagttccaccactttagactgtcatgctaatggtgtccccgagcctcagatcacttggtttaaaaacaaccacaaaatacaacaagagcctgaactgtatacatcaacgtcaccatcgtcatcgtcaagctctccattgagctccagttctagctcaagttccagctctagttcatag

SEQ ID NO: 4 - protein, 28 aa, Homo sapiens
ELYTSTSPSSSSSSPLSSSSSSSSSSSS

SEQ ID NO: 5 - DNA, 87 bp, Homo sapiens
gaactgtatacatcaacgtcaccatcgtcatcgtcatcatcaccattgtcatcatcatcatcatcgtcatcatcatcatcatcatag

SEQ ID NO: 6 - DNA, 87 bp, Homo sapiens
gaactgtatacatcaacgtcaccatcgtcatcgtcaagctctccattgagctccagttctagctcaagttccagctctagttcatag

SEQ ID NO: 7 - protein, 24 aa, Homo sapiens
STSPSSSSSSPLSSSSSSSSSSSS

SEQ ID NO: 8 - protein, 20 aa, Homo sapiens
SSSSSSPLSSSSSSSSSSSS

SEQ ID NO: 9 - protein, 16 aa, Homo sapiens
SSPLSSSSSSSSSSSS

SEQ ID NO: 10 - protein, 12 aa, Homo sapiens
SSSSSSSSSSSS

SEQ ID NO: 11 - DNA, 75 bp, Homo sapiens
tcaacgtcaccatcgtcatcgtcatcatcaccattgtcatcatcatcatcatcgtcatcatcatcatcatcatag

SEQ ID NO: 12 - DNA, 63 bp, Homo sapiens
tcgtcatcgtcatcatcaccattgtcatcatcatcatcatcgtcatcatcatcatcatcatag

SEQ ID NO: 13 - DNA, 63 bp, Homo sapiens; identical to SEQ ID NO: 12

SEQ ID NO: 14 - DNA, 39 bp, Homo sapiens
tcatcatcatcatcatcgtcatcatcatcatcatcatag

SEQ ID NO: 15 - DNA, 75 bp, Homo sapiens
tcaacgtcaccatcgtcatcgtcaagctctccattgagctccagttctagctcaagttccagctctagttcatag

SEQ ID NO: 16 - DNA, 63 bp, Homo sapiens
tcgtcatcgtcaagctctccattgagctccagttctagctcaagttccagctctagttcatag

SEQ ID NO: 17 - DNA, 51 bp, Homo sapiens
agctctccattgagctccagttctagctcaagttccagctctagttcatag

SEQ ID NO: 18 - DNA, 39 bp, Homo sapiens
agctccagttctagctcaagttccagctctagttcatag

SEQ ID NO: 19 - DNA, 20 bp, Artificial
  forward primer
atggtcagctactgggacac

SEQ ID NO: 20 - DNA, 71 bp, Artificial
  reverse primer
ctatgaactagagctggaacttgagctagaactggagctcaatggagagcttgacgatgacgatggtgacg

SEQ ID NO: 21 - protein, 15 aa, Artificial
  CESS epitope
CELYTSTSPSSSSSS

SEQ ID NO: 22 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

SEQ ID NO: 23 - DNA, 24 bp, Artificial
  Fragment 1 NUC
agctccagttctagctcaagttcc

SEQ ID NO: 24 - protein, 8 aa, Artificial
  Fragment 1 amino acid sequence
SSSSSSSS

SEQ ID NO: 25 - DNA, 24 bp, Artificial
  Fragment 2 NUC
ttgagctccagttctagctcaagt

SEQ ID NO: 26 - protein, 8 aa, Artificial
  Fragment 2 amino acid sequence
LSSSSSSS

SEQ ID NO: 27 - DNA, 24 bp, Artificial
  Fragment 3 NUC
ccattgagctccagttctagctca

SEQ ID NO: 28 - protein, 8 aa, Artificial
  Fragment 3 amino acid sequence
PLSSSSSS

SEQ ID NO: 29 - DNA, 24 bp, Artificial
  Fragment 4 NUC
tctccattgagctccagttctagc

SEQ ID NO: 30 - protein, 8 aa, Artificial
  Fragment 4 amino acid sequence
SPLSSSSS

SEQ ID NO: 31 - DNA, 24 bp, Artificial
  Fragment 5 NUC
agctctccattgagctccagttct

SEQ ID NO: 32 - protein, 8 aa, Artificial
  Fragment 5 amino acid sequence
SSPLSSSS

SEQ ID NO: 33 - DNA, 24 bp, Artificial
  Fragment 6 NUC
tcaagctctccattgagctccagt

SEQ ID NO: 34 - protein, 8 aa, Artificial
  Fragment 6 amino acid sequence
SSSPLSSS

SEQ ID NO: 35 - DNA, 24 bp, Artificial
  Fragment 7 NUC
tcgtcaagctctccattgagctcc

SEQ ID NO: 36 - protein, 8 aa, Artificial
  Fragment 7 amino acid sequence
SSSSPLSS

SEQ ID NO: 37 - DNA, 24 bp, Artificial
  Fragment 8 NUC
tcatcgtcaagctctccattgagc

SEQ ID NO: 38 - protein, 8 aa, Artificial
  Fragment 8 amino acid sequence
SSSSSPLS

SEQ ID NO: 39 - DNA, 24 bp, Artificial
  Fragment 9 NUC
tcgtcatcgtcaagctctccattg

SEQ ID NO: 40 - protein, 8 aa, Artificial
  Fragment 9 amino acid sequence
SSSSSSPL

SEQ ID NO: 41 - DNA, 24 bp, Artificial
  Fragment 10 NUC
ccatcgtcatcgtcaagctctcca

SEQ ID NO: 42 - protein, 8 aa, Artificial
  Fragment 10 amino acid sequence
PSSSSSSP

SEQ ID NO: 43 - DNA, 20 bp, Artificial
  Reverse primer for sFlt1-14 amplification
cttggctctccaactaaagg